SEQUENCE LISTING (20 sequences)

SEQ ID NO: 1 - protein, 627 aa, synthetic construct
  REGION 1..627: note = Synthetic: amino acid sequence of olipudase alfa, including its leader sequence
MARYGASLRQSCPRSGREQGQDGTAGAPGLLWMGLALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 2 - protein, 570 aa, synthetic construct
  REGION 1..570: note = Synthetic: mature olipudase alfa sequence, without leader sequence
HPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 3 - protein, 629 aa, synthetic construct
  REGION 1..629: note = Synthetic: human ASM
MPRYGASLRQSCPRSGREQGQDGTAGAPGLLWMGLVLALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESIPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSRSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 4 - protein, 570 aa, synthetic construct
  REGION 1..570: note = Synthetic: human ASM mature protein, without leader sequence
HPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESIPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSRSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 5 - protein, 629 aa, synthetic construct [sequence omitted]

SEQ ID NO: 6 - protein, 570 aa, synthetic construct; identical to SEQ ID NO: 2
  REGION 1..570: note = Synthetic: human ASM mature protein, without leader sequence

SEQ ID NO: 7 - protein, 28 aa, synthetic construct
  REGION 1..28: note = Synthetic: rhASM isoform
DSPALCRHLMPDGSLPEAQSLWPRPLFC

SEQ ID NO: 8 - protein, 28 aa, synthetic construct
  REGION 1..28: note = Synthetic: rhASM isoform with S-glutathionylation
  MOD_RES 28: note = S-glutathionylation
DSPALCRHLMPDGSLPEAQSLWPRPLFC

SEQ ID NO: 9 - protein, 28 aa, synthetic construct
  REGION 1..28: note = Synthetic: rhASM isoform with cysteinylation
  MOD_RES 28: note = cysteinylation
DSPALCRHLMPDGSLPEAQSLWPRPLFC

SEQ ID NO: 10 - protein, 27 aa, synthetic construct
  REGION 1..27: note = Synthetic: C-terminal Truncation form 1
DSPALCRHLMPDGSLPEAQSLWPRPLF

SEQ ID NO: 11 - protein, 26 aa, synthetic construct
  REGION 1..26: note = Synthetic: C-terminal Truncation form 2
DSPALCRHLMPDGSLPEAQSLWPRPL

SEQ ID NO: 12 - protein, 25 aa, synthetic construct
  REGION 1..25: note = Synthetic: C-terminal Truncation form 3
DSPALCRHLMPDGSLPEAQSLWPRP

SEQ ID NO: 13 - protein, 17 aa, synthetic construct
  REGION 1..17: note = Synthetic: C-terminus amino acid of rhASM with S-glutathionylation
  MOD_RES 17: note = S-glutathionylation
DGSLPEAQSLWPRPLFC

SEQ ID NO: 14 - protein, 17 aa, synthetic construct
  REGION 1..17: note = Synthetic: C-terminus amino acid of rhASM with cysteinylation
  MOD_RES 17: note = cysteinylation
DGSLPEAQSLWPRPLFC

SEQ ID NO: 15 - protein, 16 aa, synthetic construct
  REGION 1..16: note = Synthetic: C-terminus amino acid of rhASM with C-terminal Truncation
DGSLPEAQSLWPRPLF

SEQ ID NO: 16 - protein, 15 aa, synthetic construct
  REGION 1..15: note = Synthetic: C-terminus amino acid of rhASM with C-terminal Truncation
DGSLPEAQSLWPRPL

SEQ ID NO: 17 - protein, 14 aa, synthetic construct
  REGION 1..14: note = Synthetic: C-terminus amino acid of rhASM with C-terminal Truncation
DGSLPEAQSLWPRP

SEQ ID NO: 18 - protein, 17 aa, synthetic construct
  REGION 1..17: note = Synthetic: C-terminus amino acid of rhASM with MMTS labeling
  MOD_RES 17: note = MMTS labeling
DGSLPEAQSLWPRPLFC

SEQ ID NO: 19 - protein, 17 aa, synthetic construct
  REGION 1..17: note = Synthetic: C-terminus amino acid of rhASM having C-terminus dimerization
DGSLPEAQSLWPRPLFC

SEQ ID NO: 20 - protein, 11 aa, synthetic construct
  REGION 1..11: note = Synthetic: C-terminus amino acid of rhASM having C-terminus dimerization
DSPALCRHLMP

(19) United States
(12) Patent Application Publication
(10) Pub. No.: US 2023/0365946 A1
(43) Pub. Date: Nov. 16, 2023

(54) Pharmaceutical Recombinant Human Acid Sphingomyelinase Compositions and Methods

(71) Applicant: Genzyme Corporation, Cambridge, MA (US)

(72) Inventors: Thomas M. Wasylenko, Ashland, MA (US); Kevin Brower, Holliston, MA (US); Xiaoying Jin, Brighton, MA (US)

(73) Assignee: Genzyme Corporation, Cambridge, MA (US)

(21) Appl. No.: 18/186,731

(22) Filed: Mar. 20, 2023

Related U.S. Application Data
(60) Provisional application No. 63/321,636, filed on Mar. 18, 2022.

(51) Int. Cl. C12N 9/16; A61P 3/00; B01D 15/36; B01D 15/38; B01D 15/18; B01D 15/20

(52) U.S. Cl. CPC C12N 9/16; C12Y 301/04012; A61P 3/00; B01D 15/362; B01D 15/3828; B01D 15/1871; B01D 15/203; A61K 38/00

(57) ABSTRACT

Disclosed here are compositions comprising recombinant acid sphingomyelinase (rASM) having desired purity, specific activity, and/or rASM isoforms. Also provided are methods for making and purifying such compositions, comprising chromatography steps. Further provided are methods of modulating rASM specific activity in a composition, and methods of modulating rASM isoforms in a composition. The methods disclosed here can be particularly useful for manufacturing pharmaceutical compositions comprising rASM for treating acid sphingomyelinase deficiency (ASMD).

### CROSS-REFERENCE TO RELATED APPLICATIONS

This application claims priority from U.S. Provisional Patent Application 63/321,636, filed Mar. 18, 2022, the disclosure of which is incorporated by reference herein in its entirety.

### FIELD OF THE INVENTION

The present invention relates to compositions and methods for manufacturing recombinant acid sphingomyelinase, such as recombinant human acid sphingomyelinase.

### SEQUENCE LISTING

The instant application contains a Sequence Listing which has been submitted electronically in XML format and is hereby incorporated by reference in its entirety. The electronic copy of the Sequence Listing, created on Mar. 20, 2023, is named 022548.US042.xml and is 32,066 bytes in size.

### BACKGROUND

Acid sphingomyelinase deficiency (ASMD) is a rare life-threatening lysosomal storage disorder. It is an autosomal recessive genetic disease that results from mutations in the SMPD1 gene encoding the lysosomal enzyme acid sphingomyelinase (ASM) (Schuchman et al., Mol Genet Metab. (2017) 120(1-2):27-33). ASMD patients are unable to metabolize sphingomyelin, which as a result accumulates in lysosomes in multiple organs, causing visceral disease and neurodegeneration in severe cases. ASMD patients have increased cholesterol and other lipids in spleen, liver, lung, and bone marrow.

Olipudase alfa is a recombinant human acid sphingomyelinase, capable of significantly improving critical manifestations of ASMD in both adult and pediatric patients. However, there remains a need to produce pharmaceutical compositions comprising olipudase alfa at a commercial scale with desired purity and consistent specific activity.

### SUMMARY OF THE INVENTION

The present disclosure provides a method of purifying recombinant acid sphingomyelinase (rASM). In some embodiments, the method comprises (i) subjecting a protein mixture comprising rASM and host cell proteins (HCPs) to a cation exchange (CEX) chromatography; or subjecting a protein mixture comprising rASM and HCPs to an immobilized metal affinity chromatography (IMAC); or subjecting a protein mixture comprising rASM and HCPs to both a CEX chromatography and an IMAC. In some embodiments, the method further comprises (ii) collecting eluate from the CEX chromatography or the IMAC, thereby obtaining a purified rASM preparation.

In some embodiments, the protein mixture is subjected to a CEX chromatography and an IMAC in tandem, and eluate obtained from the CEX chromatography is subjected to the IMAC.

In some embodiments, the rASM is a recombinant human acid sphingomyelinase (rhASM).

In some embodiments, the protein mixture is obtained from Chinese Hamster Ovary (CHO) cells expressing the rASM.

In some embodiments, the rASM comprises the amino acid sequence of SEQ ID NO: 1 or SEQ ID NO: 2.

In some embodiments, the cation exchange chromatography comprising a resin selected from the group consisting of carboxymethyl (CM), sulfoethyl (SE), sulfopropyl (SP), phosphate (P) and sulfonate (S).

In some embodiments, the IMAC is a chelating resin.

In some embodiments, the IMAC is performed with zinc, copper, or nickel.

In some embodiments, the CEX chromatography comprises washing the CEX chromatography column with a CEX wash buffer having a first optimal pH and a first optimal salt concentration, wherein the first optimal pH and the first optimal salt concentration are predetermined depending on the resin and starting specific activity of the protein mixture.

In some embodiments, the CEX chromatography further comprises eluting the CEX chromatography column with a CEX elution buffer having a second optimal pH and a second optimal salt concentration, wherein under the second optimal pH and the second optimal salt concentration rASM binding on the CEX chromatography column after the washing step are removed from the column.

In some embodiments, the CEX wash buffer is selected from Table 2a, and the CEX elution buffer is selected from Table 2b.

In some embodiments, the IMAC comprises washing the IMAC column with at least one IMAC wash buffer having a third optimal pH and a third optimal salt concentration, wherein the third optimal pH and the third optimal salt concentration are predetermined depending on the resin and starting specific activity of the protein mixture.

In some embodiments, the IMAC further comprises eluting the IMAC column with an IMAC elution buffer having a fourth optimal pH and a fourth optimal salt concentration, wherein under the fourth optimal pH and the fourth optimal salt concentration rASM binding on the IMAC column after the washing step are removed from the column.

In some embodiments, the IMAC wash buffer is selected from Table 3, and the IMAC elution buffer is selected from Table 4.

In some embodiments, the purified rASM preparation has a specific activity of about 5 to 50 U/mg. In some embodiments, the purified rASM preparation has a specific activity of about 10 to 45 U/mg. In some embodiments, the purified rASM preparation has a specific activity of about 10-20 U/mg. Specific activity of the purified rASM preparation is measured according to Example 4.

In some embodiments, the obtained rASM preparation has a purity of at least 90%, at least 91%, at least 92%, at least 93%, at least 94%, at least 95%, at least 96%, at least 97%, at least 98%, or at least 99%.

In some embodiments, the obtained rASM preparation has a host cell protein (HCP) level not more than 1.0 μg/mg, not more than 2.0 μg/mg, not more than 3.0 μg/mg, not more than 4.0 μg/mg, or not more than 5.0 μg/mg.

In some embodiments, the purified rASM preparation comprises rASM isoforms with modifications in total no more than 5%, 10%, 15%, 20%, 25%, 30%, 35%, or 40% of the whole rASM population.

In some embodiments, the rASM isoforms with modifications comprise one or more modifications selected from the group consisting of C-terminus cysteinylation, S-glutathionylation, dimerization, and truncation.

In some embodiments, the protein mixture is produced in a bioreactor having a production scale of at least 100 L.

In some embodiments, the protein mixture is produced in a bioreactor having a production scale of at least 500 L.

In some embodiments, the method is conducted partially or fully under refrigerated condition at 8±3° C. In some embodiments, the method is conducted partially or fully under ambient temperature.

Also provided herein is a method of modulating the relative amounts of isoforms of recombinant acid sphingomyelinase (rASM) in an initial rASM composition, wherein the initial rASM composition comprises an unmodified rASM isoform, and at least one rASM isoform having one or more modifications selected from the group consisting of C-terminus cysteinylation, S-glutathionylation, dimerization, and truncation. In some embodiments, the method comprises subjecting the initial rASM composition to a cation exchange (CEX) chromatography; or subjecting the initial rASM composition to an immobilized metal affinity chromatography (IMAC); or subjecting the initial rASM composition to both a CEX chromatography and an IMAC. In some embodiments, the method further comprises collecting eluate from the CEX chromatography or the IMAC, thereby obtaining a purified rASM preparation.

In some embodiments, the initial rASM composition is subjected to a CEX chromatography and an IMAC in tandem, and eluate obtained from the CEX chromatography is subjected to the IMAC.

In some embodiments, the initial rASM composition is subjected to an IMAC and a CEX chromatography in tandem, and eluate obtained from the IMAC is subjected to the CEX.

In some embodiments, the initial rASM composition is subjected to both the CEX chromatography and the IMAC separately, with one or more additional steps in between.

In some embodiments, the rASM is a recombinant human acid sphingomyelinase (rhASM). In some embodiments, the rASM comprises the amino acid sequence of SEQ ID NO: 1 or SEQ ID NO: 2.

In some embodiments, the cation exchange chromatography comprises a resin selected from the group consisting of carboxymethyl (CM), sulfoethyl (SE), sulfopropyl (SP), phosphate (P) and sulfonate (S).

In some embodiments, the IMAC is a chelating resin column.

In some embodiments, the IMAC is performed with zinc, copper, or nickel.

In some embodiments, the CEX chromatography comprises washing the CEX chromatography column with a CEX wash buffer having a first optimal pH and a first optimal salt concentration, wherein the first optimal pH and the first optimal salt concentration are predetermined depending on the resin and starting specific activity of the protein mixture.

In some embodiments, the CEX chromatography further comprises eluting the CEX chromatography column with a CEX elution buffer having a second optimal pH and a second optimal salt concentration, wherein under the second optimal pH and the second optimal salt concentration all species binding on the CEX chromatography column after the washing step are removed from the column.

In some embodiments, the CEX wash buffer is selected from Table 2a, and the CEX elution buffer is selected from Table 2b.

In some embodiments, the IMAC comprises washing the IMAC column with at least one IMAC wash buffer having a third optimal pH and a third optimal salt concentration, wherein the third optimal pH and the third optimal salt concentration are predetermined depending on the resin and starting specific activity of the protein mixture.

In some embodiments, the IMAC further comprises eluting the IMAC column with an IMAC elution buffer having a fourth optimal pH and a fourth optimal salt concentration, wherein under the fourth optimal pH and the fourth optimal salt concentration all species binding on the IMAC column after the washing step are removed from the column.

In some embodiments, the IMAC wash buffer is selected from Table 3, and the IMAC elution buffer is selected from Table 4.

In some embodiments, the obtained rASM preparation has a specific activity of about 5 to 50 U/mg. In some embodiments, the obtained rASM preparation has a specific activity of about 10 to 45 U/mg. In some embodiments, the obtained rASM preparation has a specific activity of about 10 to 20 U/mg. Specific activity of the purified rASM preparation is measured according to Example 4.

In some embodiments, the obtained rASM preparation has a host cell protein (HCP) level not more than 1.0 μg/mg, not more than 2.0 μg/mg, not more than 3.0 μg/mg, not more than 4.0 μg/mg, or not more than 5.0 μg/mg.

In some embodiments, the purified rASM preparation comprises rASM isoforms with modifications in total no more than 5%, 10%, 15%, 20%, 25%, 30%, 35%, or 40% of the whole rASM population.

In some embodiments, the modifications are selected from the group consisting of C-terminus cysteinylation, S-glutathionylation, dimerization, and truncation.

In some embodiments, the initial composition comprising rASM is produced in a bioreactor having a production scale of at least 100 L or at least 500 L.

In some embodiments, the method is conducted partially or fully under refrigerated condition at 8±3° C. In some embodiments, the method is conducted partially or fully under ambient temperature.

Further provided is a method of modulating recombinant acid sphingomyelinase (rASM) specific activity in a liquid composition comprising an unmodified rASM isoform, and at least one rASM isoform having one or more modifications selected from the group consisting of C-terminus cysteinylation, S-glutathionylation, dimerization, and truncation. In some embodiments, the method comprises subjecting the liquid composition to a cation exchange (CEX) chromatography; or subjecting the liquid composition to an immobilized metal affinity chromatography (IMAC); or subjecting the liquid composition to both a CEX chromatography and an IMAC. In some embodiments, the method further comprises collecting eluate from the CEX chromatography or the IMAC, thereby obtaining a purified rASM preparation.

In some embodiments, the liquid composition is subjected to a CEX chromatography and an IMAC in tandem, and eluate obtained from the CEX chromatography is subjected to the IMAC, or wherein the liquid composition is subjected to an IMAC and a CEX chromatography in tandem, and eluate obtained from the IMAC is subjected to the CEX chromatography.

In some embodiments, the initial rASM composition is subjected to both the CEX chromatography and the IMAC separately, with one or more additional steps in between.

In some embodiments, the rASM is a recombinant human acid sphingomyelinase (rhASM). In some embodiments, the rASM comprises the amino acid sequence of SEQ ID NO: 1 or SEQ ID NO: 2.

In some embodiments, the cation exchange chromatography comprises a resin selected from the group consisting of carboxymethyl (CM), sulfoethyl (SE), sulfopropyl (SP), phosphate (P) and sulfonate (S).

In some embodiments, the IMAC is a chelating resin column.

In some embodiments, the IMAC is performed with zinc, copper, or nickel.

In some embodiments, the CEX chromatography comprises washing the CEX chromatography column with a CEX wash buffer having a first optimal pH and a first optimal salt concentration, wherein the first optimal pH and the first optimal salt concentration are predetermined depending on the resin and starting specific activity of the protein mixture.

In some embodiments, the CEX chromatography further comprises eluting the CEX chromatography column with a CEX elution buffer having a second optimal pH and a second optimal salt concentration, wherein under the second optimal pH and the second optimal salt concentration all species binding on the CEX chromatography column after the washing step are removed from the column.

In some embodiments, the CEX wash buffer is selected from Table 2a, and the CEX elution buffer is selected from Table 2b.

In some embodiments, the IMAC comprises washing the IMAC column with at least one IMAC wash buffer having a third optimal pH and a third optimal salt concentration, wherein the third optimal pH and the third optimal salt concentration are predetermined depending on the resin and starting specific activity of the protein mixture.

In some embodiments, the IMAC further comprises eluting the IMAC column with an IMAC elution buffer having a fourth optimal pH and a fourth optimal salt concentration, wherein under the fourth optimal pH and the fourth optimal salt concentration all species binding on the IMAC column after the washing step are removed from the column.

In some embodiments, the obtained rASM preparation has a specific activity of about 5 to 50 U/mg. In some embodiments, the obtained rASM preparation has a specific activity of about 10 to 45 U/mg. In some embodiments, the obtained rASM preparation has a specific activity of about 10 to 20 U/mg. Specific activity of the purified rASM preparation is measured according to Example 4.

In some embodiments, rASM in the preparation has a purity of at least 90%, at least 91%, at least 92%, at least 93%, at least 94%, at least 95%, at least 96%, at least 97%, at least 98%, or at least 99%.

In some embodiments, the obtained rASM preparation has a host cell protein (HCP) level not more than 1.0 μg/mg, not more than 2.0 μg/mg, not more than 3.0 μg/mg, not more than 4.0 μg/mg, or not more than 5.0 μg/mg.

In some embodiments, the purified rASM preparation comprises rASM isoforms with modifications in total no more than 5%, 10%, 15%, 20%, 25%, 30%, 35%, or 40% of the whole rASM population.

In some embodiments, the modifications are selected from the group consisting of C-terminus cysteinylation, S-glutathionylation, dimerization, and truncation.

In some embodiments, the liquid composition comprising rASM is produced in a bioreactor having a production scale of at least 100 L or at least 500 L.

In some embodiments, the method is conducted partially or fully under refrigerated condition at 8±3° C. In some embodiments, the method is conducted partially or fully under ambient temperature.

The present disclosure also provides a recombinant acid sphingomyelinase (rASM) preparation comprising an unmodified rASM isoform and at least one rASM isoform species having one or more modifications selected from the group consisting of C-terminus cysteinylation, S-glutathionylation, dimerization, and truncation. In some embodiments, the unmodified rASM isoform is at least 50%, 55%, 60%, 65%, 70%, 75%, or 80% of the total rASM population in the rASM preparation. In some embodiments, the unmodified rASM isoform is at least 85%, at least 86%, at least 87%, at least 88%, at least 89%, at least 90%, at least 91%, at least 92%, at least 93%, at least 94%, at least 95% or more of the total rASM population in the rASM preparation.

In some embodiments, all modified rASM isoforms in total are no more than 10%, no more than 15%, no more than 20%, no more than 25%, no more than 30%, no more than 35%, or no more than 40% of the total rASM population in the rASM preparation.

In some embodiments, all modified rASM isoforms in total are no more than 40%, no more than 35%, no more than 30%, no more than 25%, no more than 20%, no more than 15%, no more than 14%, no more than 13%, no more than 12%, no more than 11%, no more than 10%, no more than 9%, no more than 8%, no more than 7%, no more than 6%, no more than 5% or less of the total rASM population in the rASM preparation.

In some embodiments, the rASM isoform having C-terminus cysteinylation is no more than 10% of the total rASM population in the rASM preparation. In some embodiments, the rASM isoform having C-terminus cysteinylation is no more than 9%, no more than 8%, no more than 7%, no more than 6%, no more than 5% or less of the total rASM population in the rASM preparation.

In some embodiments, the rASM isoform having C-terminus S-glutathionylation is no more than 5% of the total rASM population in the rASM preparation. In some embodiments, the rASM isoform having C-terminus S-glutathionylation is no more than 5%, no more than 4%, no more than 3%, no more than 2%, no more than 1% or less of the total rASM population in the rASM preparation.

In some embodiments, the rASM isoform having C-terminus dimerization is no more than 0.2% of the total rASM population in the rASM preparation.

In some embodiments, the rASM isoform having C-terminus S-dimerization is no more than 0.1% of the total rASM population in the rASM preparation.

In some embodiments, the rASM isoform having C-terminus truncation is no more than 8% of the total rASM population in the rASM preparation.

In some embodiments, the rASM isoform having C-terminus truncation is no more than 7%, no more than 6%, no more than 5%, no more than 4%, no more than 3% or less the total rASM population in the rASM preparation.

In some embodiments, the rASM preparation has a purity of at least 95%, at least 96%, at least 97%, at least 98%, at least 99%, or more.

In some embodiments, the rASM preparation has a specific activity of about 5 to 50 U/mg. In some embodiments, rASM preparation has a specific activity of about 10 to 20 U/mg. Specific activity of the purified rASM preparation is measured according to Example 4.

In some embodiments, rASM in the preparation has a purity of at least 90%, at least 91%, at least 92%, at least 93%, at least 94%, at least 95%, at least 96%, at least 97%, at least 98%, or at least 99%.

In some embodiments, the obtained rASM preparation has a host cell protein (HCP) level not more than 1.0 μg/mg, not more than 2.0 μg/mg, not more than 3.0 μg/mg, not more than 4.0 μg/mg, or not more than 5.0 μg/mg.

In some embodiments, the rASM preparation was manufactured using a method as described herein.

Also provided is a pharmaceutical composition prepared by using the recombinant acid sphingomyelinase (rASM) preparation as described herein.

Further provided is a method of treating acid sphingomyelinase deficiency in a subject in need thereof, comprising administering the pharmaceutical composition as described herein to the subject. In some embodiments, the method further comprises a step to buffer exchange the purified rASM.

### BRIEF DESCRIPTION OF THE DRAWINGS

FIG. 1 depicts different isoforms of rhASM with or without modifications.

FIG. 2 (SEQ ID NOs: 13-20) depicts the structure near the C-terminus of rhASM with or without modifications. C-terminal status of rhASM was monitored by LC-MS of rhASM native Asp-N digests after MMTS labeling. Only C-terminus amino acids of rhASM are shown.

FIG. 3 depicts specific activity of enriched monomer of rhASM and enriched dimer of rhASM. Percent purity for each enriched population is shown above the respective bar.

FIG. 4 depicts the specific activity of rASM compositions comprising various relative abundances of total C-terminal modifications.

FIG. 5 depicts HCP clearance (upper panels) or recovery rate (lower panels) under different salt (NaCl) and pH conditions in the CEX chromatography step.

FIG. 6 depicts contour plots for HCP clearance (left panel) and recovery rate (right panel) under different salt (NaCl) and pH conditions in the IMAC step.

FIG. 7 depicts HCP clearance (upper panels) or specific activity (lower panels) under different salt (NaCl) and pH conditions in the CEX chromatography step.

FIG. 8 depicts contour plots for HCP clearance (left panel) and specific activity (right panel) under different salt (NaCl) and pH conditions in the IMAC step.

FIG. 9 depicts representative specific activity of rhASM in load material, wash fractions, and eluate fractions of the CEX operation. A number of wash conditions were tested as specified (sodium chloride 45 mM, at pH 6.3, pH 6.5, and pH 6.7).

FIG. 10A depicts specific activity of rhASM in load material, wash fractions, and eluate fractions of the IMAC process.

FIG. 10B depicts purity of rhASM in load material, wash fractions, and eluate fractions of the IMAC process. A number of wash conditions were tested (mild: 10 mM sodium phosphate at pH 6.6; medium: 10 mM sodium phosphate, 20 mM sodium chloride at pH 6.0; aggressive: 10 mM sodium phosphate, 80 mM sodium chloride at pH 5.8).

FIG. 11 depicts representative normalized abundance of rhASM isoforms in the load material, wash fractions, and eluate fractions of the CEX operation. Variant 1: unmodified rhASM isoform; Variant 2: modified rhASM isoform with C-terminal cysteine cysteinylation; Variant 3: modified rhASM isoform with C-terminal S-glutathionylation; Variant 4: dimerization form 1; Variant 5: C-terminal truncation form 1. See Table 1 for details of these isoforms.

FIG. 12 depicts representative normalized abundance of rhASM isoforms in the load material, wash fractions, and eluate fractions of the IMAC operation. Variant 1: unmodified rhASM isoform; Variant 2: modified rhASM isoform with C-terminal cysteine cysteinylation; Variant 3: modified rhASM isoform with C-terminal S-glutathionylation; Variant 4: dimerization form 1; Variant 5: C-terminal truncation form 1. See Table 1 for details of these isoforms.

FIG. 13 depicts the clinical study of using purified rhASM (olipudase alfa) to treat ASMD patients.

FIG. 14 depicts percent change in % predicted DL_{CO} in patients treated with olipudase alfa or placebo. DLCO=Carbon monoxide diffusing capacity; FVC=Forced vital capacity

FIG. 15 depicts high-resolution computerized tomography (HRCT) scans of the lungs before (left panel) and after (right panel) olipudase alfa treatment. Sphingomyelin-filled macrophages are observed as ground glass opacities.

FIG. 16 depicts HSCT ground glass appearance scores (left panel) and interstitial lung disease scores (right panel) in both lungs showed mean improvements in olipudase-alfa-treated but not placebo-treated patients.

FIG. 17 depicts % change of spleen volume (left panel) and change of splenomegaly related score (right panel) in olipudase-alfa-treated but not placebo-treated patients.

FIG. 18 depicts % change of liver volume. ALT: alanine aminotransferase; AST: aspartate aminotransferase; HDL-C: high-density lipoprotein cholesterol; LDL-C: low density lipoprotein cholesterol; MN: multiples of normal.

FIG. 19 depicts percent tissue area occupied by sphingomyelin in tissue obtained from patient treated with placebo or olipudase alfa.

FIG. 20 depicts representative toluidine blue stain images of liver biopsies in patients treated with placebo or olipudase alfa (sphingomyelin appears as dark staining).

FIG. 21 depicts normalized plasma chitotriosidase in both patients treated with placebo and patients treated with olipudase alfa (left panel), and pre-infusion plasma lyso-sphingomyelin level in both populations.

### DETAILED DESCRIPTION

Before the invention is described, it is to be understood that this invention is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood.

“About” as used herein when referring to a measurable value such as an amount, a temporal duration, and the like, is meant to encompass variations of +20% or ±10%, including ±5%, ±1%, and ±0.1% from the specified value, as such variations are appropriate to perform the disclosed methods.

“Polypeptide,” “peptide” and “protein” are used interchangeably herein to refer to a polymer of amino acid residues. A polypeptide can be of natural (tissue-derived) origins, recombinant or natural expression from prokaryotic or eukaryotic cellular preparations, or produced chemically via synthetic methods. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid mimetics refers to chemical compounds that have a structure that is different from the general chemical structure of an amino acid, but that functions in a manner similar to a naturally occurring amino acid. Non-natural residues are well described in the scientific and patent literature.

“Peptide” as used herein includes peptides which are conservative variations of those peptides specifically exemplified herein. “Conservative variation” as used herein denotes the replacement of an amino acid residue by another, biologically similar residue. Examples of conservative variations include, but are not limited to, the substitution of one hydrophobic residue such as isoleucine, valine, leucine, alanine, cysteine, glycine, phenylalanine, proline, tryptophan, tyrosine, norleucine, or methionine for another, or the substitution of one polar residue for another, such as the substitution of arginine for lysine, glutamic for aspartic acids, or glutamine for asparagine, and the like. Neutral hydrophilic amino acids which can be substituted for one another include asparagine, glutamine, serine, and threonine. “Conservative variation” also includes the use of a substituted amino acid in place of an unsubstituted parent amino acid provided that antibodies raised to the substituted polypeptide also immunoreact with the unsubstituted polypeptide. Such conservative substitutions are within the definition of the classes of the peptides of the invention.

“Recombinant” when used with reference to a protein indicates that the protein has been produced by the introduction of a heterologous nucleic acid into a host cell.

“Load,” as used herein, is the composition loaded onto a chromatography material. Loading buffer is the buffer used to load the composition comprising the product of interest onto a chromatography material. The chromatography material may be equilibrated with an equilibration buffer prior to loading the composition which is to be purified. In some examples, the wash buffer is used after loading the composition onto a chromatography material and before elution of the polypeptide of interest from the solid phase. However, some of the product of interest, e.g., a polypeptide, may be removed from the chromatography material by the wash buffer (i.e. in the flow-through).

“Elution,” as used herein, is the removal of the product, e.g., polypeptide, from the chromatography material. Elution buffer is the buffer used to elute the polypeptide or other product of interest from a chromatography material. In many cases, an elution buffer has a different physical characteristic than the load buffer. For example, the elution buffer may have a different conductivity than load buffer or a different pH than the load buffer. In some embodiments, the elution buffer has a lower conductivity than the load buffer. In some embodiments, the elution buffer has a higher conductivity than the load buffer. In some embodiments, the elution buffer has a lower pH than the load buffer. In some embodiments, the elution buffer has a higher pH than the load buffer. In some embodiments, the elution buffer has a different conductivity and a different pH than the load buffer. The elution buffer can have any combination of higher or lower conductivity and higher or lower pH.

“Conductivity” refers to the ability of an aqueous solution to conduct an electric current between two electrodes. In solution, the current flows by ion transport. Therefore, with an increasing amount of ions present in the aqueous solution, the solution will have a higher conductivity. The basic unit of measure for conductivity is the Siemen (or mho), mho (mS/cm), and can be measured using a conductivity meter, such as various models of Orion conductivity meters. Since electrolytic conductivity is the capacity of ions in a solution to carry electrical current, the conductivity of a solution may be altered by changing the concentration of ions therein. For example, the concentration of a buffering agent and/or the concentration of a salt (e.g., sodium chloride, sodium acetate, or potassium chloride) in the solution may be altered in order to achieve the desired conductivity. Preferably, the salt concentration of the various buffers is modified to achieve the desired conductivity.

“Host cell proteins” (HCPs) are proteins from the cells in which the polypeptide was produced. For example, CHOP are proteins from host cells, i.e., Chinese Hamster Ovary Proteins. The amount of CHOP may be measured by enzyme-linked immunosorbent assay (“ELISA”) or mass spectrometry. In some embodiments of any of the methods described herein, the amount of HCP (e.g., CHOP) is reduced by greater than about any of 10%, 20%, 30%, 40%, 50%, 60%, 70%, 80%, 90%, or 95%. The amount of HCP may be reduced by between about any of 10% and 99%, 30% and 95%, 30% and 99%, 50% and 95%, 50% and 99%, 75% and 99%, or 85% and 99%. In some embodiments, the amount of HCP is reduced by about any of 10%, 20%, 30%, 40%, 50%, 60%, 70%, 80%, 85%, 90%, 95%, or 98%. In some embodiments, the reduction is determined by comparing the amount of HCP in the composition recovered from a purification step(s) to the amount of HCP in the composition before the purification step(s).

The present disclosure provides compositions comprising a recombinant ASM, such as a recombinant human ASM (rhASM). In some embodiments, the rhASM is olipudase alfa. The compositions of the present disclosure have superior uniformity and purity. In some embodiments, the compositions of the invention are pharmaceutical compositions, i.e., compositions that are in such a form, or can be prepared to become such a form, as to permit the biological activity of the active ingredient to be effective while containing no additional ingredients that are significantly toxic or otherwise cause unwanted side effects not related to the active ingredient in patients. The terms “pharmaceutical composition” and “pharmaceutical preparation” are used interchangeably herein. The pharmaceutical compositions of the present invention are useful in treating patients with ASM deficiency.

Drug-substance consistency is an important consideration for the final step in the manufacture of drug substance/active pharmaceutical ingredient. It ensures that a consistent efficacy is maintained between batches, thereby assuring quality. Studies are necessary to ensure that the entire contents of the batch are homogenous and consistent among batches.

During the purification process, conditions which resulted in superior host cell protein clearance may affect the specific activity of the product. The inventors carried out studies to identify critical steps to ensure that specific activity in the final product is well-controlled in the purification process. As a result, inventors discovered ways to control the proportion of rhASM isoforms (e.g., rhASM isoforms with C-terminal cysteine modifications including cysteinylation, S-glutathionylation, dimerization, and C-terminal truncation) in a rhASM product through modulating the relative amounts of unmodified rhASM and modified rhASM isoforms, thereby controlling the specific activity of the final product. The findings lead to robust and effective control of product quality and process performance.

### Recombinant Human Acid Sphingomyelinase

ASM is an enzyme catalyzing the breakdown of sphingomyelin to ceramide and phosphorylcholine. “Recombinant human ASM” refers to human ASM, with or without certain amino acid modifications relative to a wildtype sequence, that is prepared by recombinant means. For example, a recombinant human ASM may be expressed in cultured mammalian host cells (e.g., COS, CHO, HeLa, 3T3, 293T, NSO, SP2/0, or HuT 78 cells and the like) or in animals transgenic for a human ASM coding sequence.

In some embodiments, the recombinant human ASM is olipudase alfa. Olipudase alfa is the glycoform alpha of a human ASM (EC-3.1.4.12) produced in CHO cells. Mature olipudase alfa is a 570 amino acid polypeptide that retains the enzymatic and lysosomal targeting activity of the native human protein. The amino acid sequence of olipudase alfa, including its leader sequence (residues 1-57), is shown below as SEQ ID NO: 1, where the leader sequence is italicized and in boldface. The mature olipudase alfa sequence (SEQ ID NO: 2, which spans residues 58-627 of SEQ ID NO: 1) does not have the leader sequence.

(SEQ ID NO: 1)

[symbol]

[symbol]

[symbol]

[symbol]

[symbol]

HPLSPQGHPARLHRIVPRLRDVFGWGNLTCPICKGL
FTAINLGLKKEPNVARVGSVAIKLCNLLKIAPPAVCQSIVHLFEDDMVE
VWRRSVLSPSEACGLLLGSTCGHWDIFSSWNISLPTVPKPPPKPPSPPA
PGAPVSRILFLTDLHWDHDYLEGTDPDCADPLCCRRGSGLPPASRPGAG
YWGEYSKCDLPLRTLESLLSGLGPAGPFDMVYWTGDIPAHDVWHQTRQD
QLRALTTVTALVRKFLGPVPVYPAVGNHESTPVNSFPPPFIEGNHSSRW
LYEAMAKAWEPWLPAEALRTLRIGGFYALSPYPGLRLISLNMNFCSREN
FWLLINSTDPAGQLQWLVGELQAAEDRGDKVHIIGHIPPGHCLKSWSWN
YYRIVARYENTLAAQFFGHTHVDEFEVFYDEETLSRPLAVAFLAPSATT
YIGLNPGYRVYQIDGNYSGSSHVVLDHETYILNLTQANIPGAIPHWQLL
YRARETYGLPNTLPTAWHNLVYRMRGDMQLFQTFWFLYHKGHPPSEPCG
TPCRLATLCAQLSARADSPALCRHLMPDGSLPEAQSLWPRPLFC
(SEQ ID NO: 2)
HPLSPQGHPARLHRIVPRLRDVFGWGNLTCPICKGLFTAINLGLKKEPN
VARVGSVAIKLCNLLKIAPPAVCQSIVHLFEDDMVEVWRRSVLSPSEAC
GLLLGSTCGHWDIFSSWNISLPTVPKPPPKPPSPPAPGAPVSRILFLTD
LHWDHDYLEGTDPDCADPLCCRRGSGLPPASRPGAGYWGEYSKCDLPLR
TLESLLSGLGPAGPFDMVYWTGDIPAHDVWHQTRQDQLRALTTVTALVR
KFLGPVPVYPAVGNHESTPVNSFPPPFIEGNHSSRWLYEAMAKAWEPWL
PAEALRTLRIGGFYALSPYPGLRLISLNMNFCSRENFWLLINSTDPAGQ
LQWLVGELQAAEDRGDKVHIIGHIPPGHCLKSWSWNYYRIVARYENTLA
AQFFGHTHVDEFEVFYDEETLSRPLAVAFLAPSATTYIGLNPGYRVYQI
DGNYSGSSHVVLDHETYILNLTQANIPGAIPHWQLLYRARETYGLPNTL
PTAWHNLVYRMRGDMQLFQTFWFLYHKGHPPSEPCGTPCRLATLCAQLS
ARADSPALCRHLMPDGSLPEAQSLWPRPLFC

In other embodiments, the human ASM useful in the present invention is 99%, 98%, 97%, 96%, or 95% identical in amino acid sequence to olipudase alfa. For example, the human ASM in the composition may have the sequence shown in U.S. Pat. No. 6,541,218, the disclosure of which is incorporated by reference herein in its entirety. That sequence (SEQ ID NO: 3) is shown below, with the leader sequence (residues 1-59) italicized and in boldface, where the mature protein (SEQ ID NO: 4, which spans residues 60-629 of SEQ ID NO: 3) does not have the leader sequence.

(SEQ ID NO: 3)

[symbol]

[symbol]

[symbol]

[symbol]

[symbol]

HPLSPQGHPARLHRIVPRLRDVFG
WGNLTCPICKGLFTAINLGLKKEPNVARVGSVAIKLCNLLKIAPPAVCQ
SIVHLFEDDMVEVWRRSVLSPSEACGLLLGSTCGHWDIFSSWNISLPTV
PKPPPKPPSPPAPGAPVSRILFLTDLHWDHDYLEGTDPDCADPLCCRRG
SGLPPASRPGAGYWGEYSKCDLPLRTLESLLSGLGPAGPFDMVYWTGDI
PAHDVWHQTRQDQLRALTTVTALVRKFLGPVPVYPAVGNHESIPVNSFP
PPFIEGNHSSRWLYEAMAKAWEPWLPAEALRTLRIGGFYALSPYPGLRL
ISLNMNFCSRENFWLLINSTDPAGQLQWLVGELQAAEDRGDKVHIIGHI
PPGHCLKSWSWNYYRIVARYENTLAAQFFGHTHVDEFEVFYDEETLSRP
LAVAFLAPSATTYIGLNPGYRVYQIDGNYSRSSHVVLDHETYILNLTQA
NIPGAIPHWQLLYRARETYGLPNTLPTAWHNLVYRMRGDMQLFQTFWFL
YHKGHPPSEPCGTPCRLATLCAQLSARADSPALCRHLMPDGSLPEAQSL
WPRPLFC
(SEQ ID NO: 4)
HPLSPQGHPARLHRIVPRLRDVFGWGNLTCPICKGLFTAINLGLKKEPN
VARVGSVAIKLCNLLKIAPPAVCQSIVHLFEDDMVEVWRRSVLSPSEAC
GLLLGSTCGHWDIFSSWNISLPTVPKPPPKPPSPPAPGAPVSRILFLTD
LHWDHDYLEGTDPDCADPLCCRRGSGLPPASRPGAGYWGEYSKCDLPLR
TLESLLSGLGPAGPFDMVYWTGDIPAHDVWHQTRQDQLRALTTVTALVR
KFLGPVPVYPAVGNHESIPVNSFPPPFIEGNHSSRWLYEAMAKAWEPWL
PAEALRTLRIGGFYALSPYPGLRLISLNMNFCSRENFWLLINSTDPAGQ
LQWLVGELQAAEDRGDKVHIIGHIPPGHCLKSWSWNYYRIVARYENTLA
AQFFGHTHVDEFEVFYDEETLSRPLAVAFLAPSATTYIGLNPGYRVYQI
DGNYSRSSHVVLDHETYILNLTQANIPGAIPHWQLLYRARETYGLPNTL
PTAWHNLVYRMRGDMQLFQTFWFLYHKGHPPSEPCGTPCRLATLCAQLS
ARADSPALCRHLMPDGSLPEAQSLWPRPLFC

The human ASM in the composition may also be identical in amino acid sequence to the human ASM disclosed in the UNIPROT database as sequence P17405-1, or polymorphic variants thereof. The P17405-1 sequence is shown below (SEQ ID NO: 5), with the leader sequence (residues 1-59) italicized and in boldface, where the mature protein (SEQ ID NO: 6, which spans residues 60-629 of SEQ ID NO: 5) does not have the leader sequence.

(SEQ ID NO: 5)

[symbol]

[symbol]

[symbol]

[symbol]

[symbol]

HPLSPQGHPARLHRIVPRLRDVFGWGNLTCPICKGLFT
AINLGLKKEPNVARVGSVAIKLCNLLKIAPPAVCQSIVHLFEDDMVEVW
RRSVLSPSEACGLLLGSTCGHWDIFSSWNISLPTVPKPPPKPPSPPAPG
APVSRILFLTDLHWDHDYLEGTDPDCADPLCCRRGSGLPPASRPGAGYW
GEYSKCDLPLRTLESLLSGLGPAGPFDMVYWTGDIPAHDVWHQTRQDQL
RALTTVTALVRKFLGPVPVYPAVGNHESTPVNSFPPPFIEGNHSSRWLY
EAMAKAWEPWLPAEALRTLRIGGFYALSPYPGLRLISLNMNFCSRENFW
LLINSTDPAGQLQWLVGELQAAEDRGDKVHIIGHIPPGHCLKSWSWNYY
RIVARYENTLAAQFFGHTHVDEFEVFYDEETLSRPLAVAFLAPSATTYI
GLNPGYRVYQIDGNYSGSSHVVLDHETYILNLTQANIPGAIPHWQLLYR
ARETYGLPNTLPTAWHNLVYRMRGDMQLFQTFWFLYHKGHPPSEPCGTP
CRLATLCAQLSARADSPALCRHLMPDGSLPEAQSLWPRPLFC
(SEQ ID NO: 6)
HPLSPQGHPARLHRIVPRLRDVFGWGNLTCPICKGLFTAINLGLKKEPN
VARVGSVAIKLCNLLKIAPPAVCQSIVHLFEDDMVEVWRRSVLSPSEAC
GLLLGSTCGHWDIFSSWNISLPTVPKPPPKPPSPPAPGAPVSRILFLTD
LHWDHDYLEGTDPDCADPLCCRRGSGLPPASRPGAGYWGEYSKCDLPLR
TLESLLSGLGPAGPFDMVYWTGDIPAHDVWHQTRQDQLRALTTVTALVR
KFLGPVPVYPAVGNHESTPVNSFPPPFIEGNHSSRWLYEAMAKAWEPWL
PAEALRTLRIGGFYALSPYPGLRLISLNMNFCSRENFWLLINSTDPAGQ
LQWLVGELQAAEDRGDKVHIIGHIPPGHCLKSWSWNYYRIVARYENTLA
AQFFGHTHVDEFEVFYDEETLSRPLAVAFLAPSATTYIGLNPGYRVYQI
DGNYSGSSHVVLDHETYILNLTQANIPGAIPHWQLLYRARETYGLPNTL
PTAWHNLVYRMRGDMQLFQTFWFLYHKGHPPSEPCGTPCRLATLCAQLS
ARADSPALCRHLMPDGSLPEAQSLWPRPLFC

rhASM DNA, diagnostic methods, and rhASM proteins are covered by U.S. Pat. Nos. 5,773,278, 5,686,240, and 6,541,218, each of which is herein incorporated by reference in its entirety.

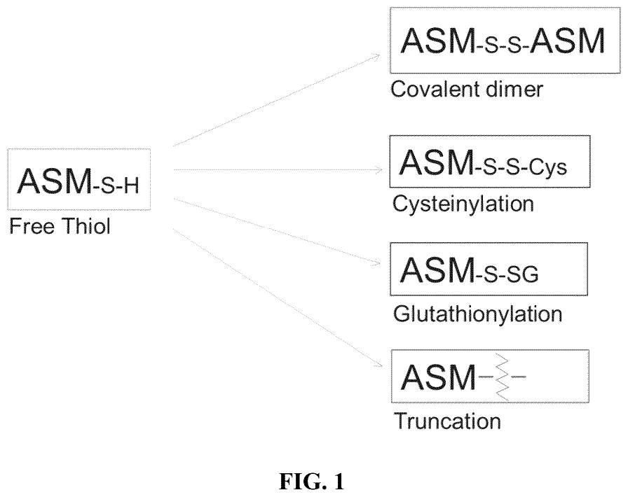

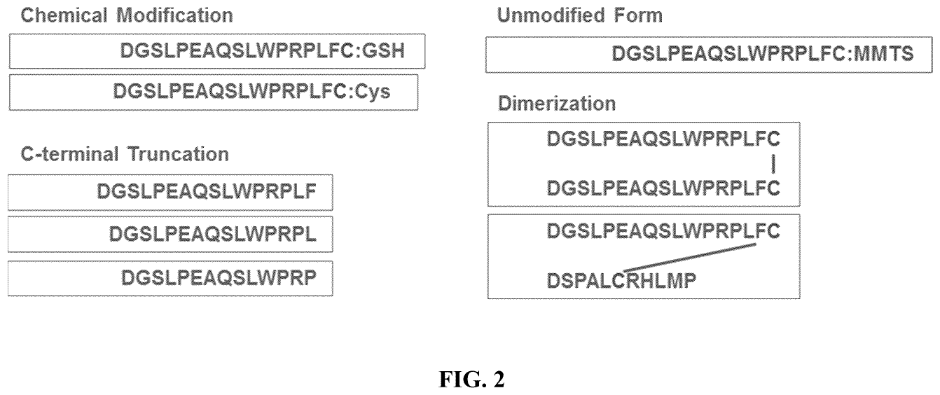

Recombinant human ASM (rhASM) produced in host cells may exist as a mixture of one or more isoforms. In some embodiments, the isoforms are demonstrated in FIG. 1 and FIG. 2, also as summarized in Table 1 below. C-terminal status and relative proportions of C-terminal modified species can be determined by LC-MS analysis.

TABLE 1
rhASM isoforms

| Isoform | C-terminal sequences only* |
|---|---|
| Unmodified form | . . . DSPALCRHLMPDGSLPEAQSLWPRPLFC (SEQ ID NO: 7) |
| (monomer having free | |
| thiol at the C-terminus) | |
| Dimerization form 1 | . . . DSPALCRHLMPDGSLPEAQSLWPRPLFC (SEQ ID NO: 7) |
| | \| |
| | . . . DSPALCRHLMPDGSLPEAQSLWPRPLFC (SEQ ID NO: 7) |
| Dimerization form 2 | . . . DSPALCRHLMPDGSLPEAQSLWPRPLFC (SEQ ID NO: 7) |
| | \| |
| | . . . DSPALCRHLMPDGSLPEAQSLWPRPLFC (SEQ ID NO: 7) |
| S-glutathionylation | . . . DSPALCRHLMPDGSLPEAQSLWPRPLFC:GSH (SEQ ID NO: 8) |
| cysteinylation | . . . DSPALCRHLMPDGSLPEAQSLWPRPLFC:Cys (SEQ ID NO: 9) |
| C-terminal Truncation | . . . DSPALCRHLMPDGSLPEAQSLWPRPLF (SEQ ID NO: 10) |
| form 1 | (C deleted) |
| C-terminal Truncation | . . . DSPALCRHLMPDGSLPEAQSLWPRPL (SEQ ID NO: 11) |
| form 2 | (FC deleted) |
| C-terminal Truncation | . . . DSPALCRHLMPDGSLPEAQSLWPRP (SEQ ID NO: 12) |
| form 3 | (LFC deleted) |

*(only showing rhASM amino acids starting at position 600 of SEQ ID NO: 1, position 543 of SEQ ID NO: 2, SEQ ID NO: 4, or SEQ ID NO: 6, or position 602 of SEQ ID NO: 3 or SEQ ID NO: 5)

Methods of Cell Culture

Compositions and methods for expression of recombinant ASM in host cells, such as Chinese hamster ovary cells, are described in U.S. Pat. No. 5,773,278, which is herein incorporated by reference in its entirety. In order to express a biologically active ASM, the coding sequence for the enzyme, a functional equivalent, or a modified sequence, can be inserted into an appropriate expression vector, i.e., a vector which contains the necessary elements for transcription and translation of the inserted coding sequence in appropriate host cells. Host cell expression systems which possess the cellular machinery and elements for proper processing, i.e., signal cleavage, glycosylation, phosphorylation, and protein sorting, can be used. For example, mammalian host cell expression systems can be used for the expression of biologically active enzymes that are properly folded and processed. When administered in humans, such expression products should exhibit proper tissue targeting and no adverse immunological reaction.

Methods which are well-known to those skilled in the art can be used to construct expression vectors containing the ASM coding sequence and appropriate transcriptional/translational control signals. These methods include in vitro recombination/genetic recombination. See, for example, the techniques described in Maniatis et al., Molecular Cloning A Laboratory Manual, Cold spring Harbor Laboratory, N.Y., Chapter 12 (1982).

In bacterial systems, a number of expression vectors may be advantageously selected depending upon the use intended for the ASM protein expressed. For example, when large quantities of ASM are to be produced, vectors which direct the expression of high levels of fusion protein products that are readily purified may be desirable. Such vectors include, but are not limited to, the E. coli expression vector pUR278 (Ruther et al., EMBO J. (1983) 2:1791), in which the ASM coding sequence may be ligated into the vector in frame with the lac Z coding region so that a hybrid AS-lac Z protein is produced; pIN vectors (Inouye & Inouye, Nucleic acids Res. (1985) 13:3101-9; Van Heeke & Schuster, J Biol Chem. (1989) 264:5503-9); and the like.

A variety of eukaryotic host-expression systems may be utilized to express the ASM coding sequence. Although prokaryotic systems offer the distinct advantage of ease of manipulation and low cost of scale-up, their major drawback in the expression of ASM is their lack of proper post-translational modifications of expressed mammalian proteins. Eukaryotic systems, and preferably mammalian expression systems, allow for proper modification to occur. Eukaryotic cells which possess the cellular machinery for proper processing of the primary transcript, e.g., glycosylation, phosphorylation, and advantageous secretion of the gene product, should be used as host cells for the expression of ASM. Mammalian cell lines are preferred. Such host cell lines may include, but are not limited to, CHO, VERO, BHK, HeLa, COS, MDCK, -293, WI38, etc.

For long-term, high-yield production of recombinant proteins, stable expression can be used. For example, following the introduction of foreign DNA, engineered cells may be allowed to grow for 1-2 days in an enriched media, and then are switched to a selective media. Rather than using expression vectors which contain viral origins of replication, host cells can be transformed with the ATN or DNA controlled by appropriate expression control elements (e.g., promoter, enhancer, sequences, transcription terminators, polyadenylation sites, etc.), and a selectable marker. The selectable marker in the recombinant plasmid confers resistance to selection and allows cells to stably integrate the plasmid into their chromosomes and grow to form foci which in turn can be cloned and expanded into cell lines. A number of selection systems may be used, including but not limited to, the herpes simplex virus thymidine kinase (Wigler et al., Cell (1977) 11:223), hypoxanthine-guanine phosphoribosyltransferase (Szybalska & Szybalski, Proc Natl Acad Sci. USA (1962) 48:2026), and adenine phosphoribosyltransferase (Lowy et al., Cell (1980) 22:817) genes can be employed in tk-, hgprt- or aprt-cells respectively. Also, antimetabolite resistance can be used as the basis of selection for dhfr, which confers resistance to methotrexate (Wigler et al., Proc Natl Acad Sci. USA (1980) 77:3567; O'Hare et al., Proc Natl Acad Sci. USA (1981) 78:1527); gpt, which confers resistance to mycophenolic acid (Mulligan & Berg, Proc Natl Acad Sci. USA (1981) 78:2072; neo, which confers resistance to the aminoglycoside G-418 (Colberre-Garapin et al., J Mol Biol. (1981) 150:1); and hygro, which confers resistance to hygromycin (Santerre et al., Gene (1984) 30:147) genes. Recently, additional selectable genes have been described, namely trpB, which allows cells to utilize indole in place of tryptophan; hisD, which allows cells to utilize histinol in place of histidine (Hartman & Mulligan, Proc Natl Acad Sci. USA (1988) 85:8047); and ODC (ornithine decarboxylase) which confers resistance to the ornithine decarboxylase inhibitor, 2-(difluoromethyl)-DL-ornithine, DFMO (McConlogue L., In: Current Communications in Molecular Biology, Cold Spring Harbor Laboratory ed. (1987)).

Alternative eukaryotic expression systems which may be used to express the ASM enzymes are yeast transformed with recombinant yeast expression vectors containing the ASM coding sequence; insect cell systems infected with recombinant virus expression vectors (e.g., baculovirus) containing the ASM coding sequence; or plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the ASM coding sequence.

In yeast, a number of vectors containing constitutive or inducible promoters may be used. For a review see, Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 31987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. For complementation assays in yeast, cDNAs for ASM may be cloned into yeast episomal plasmids (YEp) which replicate autonomously in yeast due to the presence of the yeast 2μ circle. The cDNA may be cloned behind either a constitutive yeast promoter such as ADH or LEU2 or an inducible promoter such as GAL (Cloning in Yeast, Chpt. 3, R. Rothstein In: DNA Cloning Vol. 11, A Practical Approach, Ed. D. M. Glover, 1986, IRL Press, Wash., D.C.). Constructs may contain the 5′ and 3′ non-translated regions of the cognate ASM mRNA or those corresponding to a yeast gene. YEp plasmids transform at high efficiency and the plasmids are extremely stable. Alternatively, vectors may be used which promote integration of foreign DNA sequences into the yeast chromosome.

In cases where plant expression vectors are used, the expression of the ASM coding sequence may be driven by any of a number of promoters. For example, viral promoters such as the 35S RNA and 19S RNA promoters of CaMV (Brisson et al., Nature (1984) 310:511-514), or the coat protein promoter of TMV (Takamatsu et al., EMBO J. (1987) 6:307-311) may be used; alternatively, plant promoters such as the small subunit of RUBISCO (Coruzzi et al., EMBO J. (1984) 3:1671-1680; Broglie et al., Science (1984) 224:838-843); or heat shock promoters, e.g., soybean hsp17.5-E or hsp17.3-B (Gurley et al., Mol Cell Biol. (1986) 6:559-565) may be used. These constructs can be introduced into plant cells using Ti plasmids, Ri plasmids, plant virus vectors; direct DNA transformation; microinjection, electroporation, etc. For reviews of such techniques see, for example, Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9.

An alternative expression system which could be used to express ASM is an insect system. In one such system, Autographa californica nuclear polyhedrosis virus (AcNPV) is used as a vector to express foreign genes. The virus grows in Spodoptera frugiperda cells. The ASM sequence may be cloned into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed (see, e.g., Smith et al., J Viol. (1983) 46:584; Smith, U.S. Pat. No. 4,215,051).

Methods of Purification and Modulating rASM Specific Activity and Isoforms

The present disclosure provides the unexpected discovery that by varying the purification process, one can modulate the specific activity of rASM (e.g., rhASM), and the ratio of rASM isoforms in a composition comprising rASM. This innovation can be used to adjust the specific activity of a batch of rASM into a target range. This method of adjusting the specific activity may be more straightforward to implement without significant impact to process performance relative to other potential methods (e.g., changes to the cell culture process).

Accordingly, the following methods are provided in the present disclosure:

I. Methods of purifying rASM from a protein mixture. In some embodiments, the protein mixture comprises rASM and at least another protein. In some embodiments, the protein mixture comprises rASM and host cell proteins, such as CHO cell proteins. In some embodiments, the methods comprise subjecting a protein mixture comprising rASM and host cell proteins (HCPs) to a cation exchange (CEX) chromatography as described herein. In some embodiments, the methods further comprise collecting eluate from the CEX chromatography, thereby obtaining a purified rASM preparation. In some embodiments, the methods comprise subjecting a protein mixture comprising rASM and HCPs to an immobilized metal affinity chromatography (IMAC) as described herein. In some embodiments, the methods further comprise collecting eluate from the IMAC chromatography, thereby obtaining a purified rASM preparation. In further embodiments, the methods comprise subjecting a protein mixture comprising rASM and HCPs to both a CEX chromatography and an IMAC, whether separately or in tandem. The sequence of CEX and IMAC are switchable. For example, in some embodiments, the methods comprise (i) subjecting a protein mixture comprising rASM and host cell proteins (HCPs) to a CEX chromatography as described herein. In some embodiments, the methods further comprise (ii) subjecting eluate obtained from the CEX chromatography directly or indirectly to an IMAC as described herein. As used herein, the term “directly” means that the eluate obtained from the CEX chromatography is subjected to the IMAC directly without being processed through another step, while the term “indirectly” means that the eluate obtained from the CEX chromatography is processed though one or more additional steps before it is subjected to the IMAC. For example, the eluate obtained from the CEX chromatography goes through another purification step (e.g., another purification column) before it is subjected to the IMAC. In some embodiments, the methods further comprise collecting eluate from the IMAC, thereby obtaining a purified rASM preparation. Optionally, the sequence of the CEX chromatography and IMAC can be exchanged. For example, in some embodiments, the methods comprise (i) subjecting a protein mixture comprising rASM and HCPs to an IMAC as described herein. In some embodiments, the methods further comprise (ii) subjecting eluate obtained from the IMAC directly or indirectly to a CEX chromatography as described herein. As used herein, the term “directly” means that the eluate obtained from the IMAC is subjected to the CEX chromatography directly without being processed through another step, while the term “indirectly” means that the eluate obtained from the IMAC is processed though one or more additional step before it is subjected to the CEX chromatography. For example, the eluate obtained from the IMAC goes through another purification step (e.g., another purification column) before it is subjected to the CEX chromatography. In some embodiments, the methods further comprise collecting eluate from the CEX chromatography thereby obtaining a purified rASM preparation. In some embodiments, one or more additional purification steps can be included before and/or after the sample is subjected to CEX chromatography and/or IMAC in order to remove impurities (e.g., HCPs) from a sample. Such purification steps are discussed in U.S. Pat. Nos. 8,796,419, 9,481,706, 10,259,842, and PCT Publication Nos. WO 2008/085988 A1 and WO 2019/121846 A1, each of which is incorporated by reference in its entirety.

II. Methods of modulating relative amounts of isoforms of recombinant acid sphingomyelinase (rASM) in an initial rASM composition. Relative amount of an rASM isoform in a composition is equal to the percentage of normalized abundance of the isoform compared to total rASM abundance when all rASM isoforms are combined. For example, if the normalized abundance of unmodified rASM in a composition is 9 million, while the total rASM abundance of all rASM isoforms in the composition combined together is 10 million, then the relative amount of the unmodified rASM in the composition is 90%. The initial rASM composition may comprise an unmodified rASM isoform, and at least one rASM isoform having one or more modifications selected from the group consisting of C-terminus cysteinylation, S-glutathionylation, dimerization, and truncation. In some embodiments, the methods comprise subjecting the initial rASM composition to a CEX chromatography as described herein. In some embodiments, the methods further comprise collecting the eluate from the CEX chromatography, thereby obtaining an rASM preparation with a modulated relative amount of isoforms of rASM. In some embodiments, the methods comprise subjecting the initial rASM composition to an immobilized metal affinity chromatography (IMAC) as described herein. In some embodiments, the methods further comprise collecting the eluate from the IMAC chromatography, thereby obtaining an rASM preparation with a modulated relative amount of isoforms of rASM. In further embodiments, the methods comprise subjecting the initial rASM composition to both a CEX chromatography and an IMAC, whether separately or in tandem. The sequence of CEX and IMAC are switchable. For example, in some embodiments, the methods comprise (i) subjecting the initial composition comprising rASM to a cation exchange (CEX) chromatography as described herein. In some embodiments, the methods further comprise (ii) collecting the eluate from the cation exchange (CEX) chromatography, thereby obtaining an rASM preparation with a modulated relative amount of isoforms of rASM. In some embodiments, the methods further comprise subjecting the eluate obtained from the CEX chromatography directly or indirectly to an IMAC. In some embodiments, the methods further comprise collecting the eluate from the IMAC thereby obtaining an rASM preparation with a modulated relative amount of isoforms of rASM. Optionally, the sequence of the CEX chromatography and IMAC can be exchanged. For example, in some embodiments, the methods comprise (i) subjecting the initial composition comprising rASM to an IMAC as described herein. In some embodiments, the methods further comprise (ii) collecting the eluate from the IMAC, thereby obtaining an rASM preparation with a modulated relative amount of isoforms of rASM. In some embodiments, the methods further comprise subjecting the eluate obtained from the IMAC directly or indirectly to a CEX chromatography. In some embodiments, the methods further comprise collecting the eluate from the CEX chromatography thereby obtaining an rASM preparation with a modulated relative amount of isoforms of rASM. In some embodiments, such methods increase the relative amount of an unmodified rASM isoform in the compositions, while reducing at least one relative amount of modified rASM isoforms selected from the group consisting of C-terminus cysteinylation, S-glutathionylation, dimerization, and truncation, as described herein. In some embodiments, the relative amount of the unmodified rASM isoform in the obtained compositions when compared to that in the initial rASM composition is increased by at least 0.1%, at least 0.2%, at least 0.3%, at least 0.4%, at least 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, at least 2.0%, at least 2.1%, at least 2.2%, at least 2.3%, at least 2.4%, at least 2.5%, at least 2.6%, at least 2.7%, at least 2.8%, at least 2.9%, at least 3.0%, at least 3.1%, at least 3.2%, at least 3.3%, at least 3.4%, at least 3.5%, at least 3.6%, at least 3.7%, at least 3.8%, at least 3.9%, at least 4.0%, at least 4.1%, at least 4.2%, at least 4.3%, at least 4.4%, at least 4.5%, at least 4.6%, at least 4.7%, at least 4.8%, at least 4.9%, at least 5.0%, or more. In some embodiments, the relative amount of a modified rASM isoform is reduced by at least 5%, at least 10%, 15%, at least 20%, 25%, at least 30%, at least 35%, at least 40%, at least 45%, at least 50%, at least 55%, at least 60%, at least 65%, at least 70%, at least 75%, at least 80%, at least 85%, at least 90%, or more. In some embodiments, the relative amount of the unmodified rASM isoform in the obtained composition is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, or more.

III. Methods of modulating recombinant acid sphingomyelinase (rASM) specific activity in a liquid composition comprising an unmodified rASM isoform, and at least one rASM isoform having one or more modifications selected from the group consisting of C-terminus cysteinylation, S-glutathionylation, dimerization, and truncation. In some embodiments, the methods comprise subjecting the liquid composition to a cation exchange (CEX) chromatography as described herein. In some embodiments, the methods further comprise (ii) collecting eluate from the cation exchange (CEX) chromatography, thereby obtaining an rASM preparation with modulated specific activity. In some embodiments, the methods comprise subjecting the liquid composition to an immobilized metal affinity chromatography (IMAC) as described herein. In some embodiments, the methods further comprise collecting eluate from the IMAC chromatography, thereby obtaining an rASM preparation with modulated specific activity. In further embodiments, the methods comprise subjecting the initial rASM composition to both a CEX chromatography and an IMAC, whether separately or in tandem. The sequence of CEX and IMAC are switchable. For example, in some embodiments, the methods comprise (i) subjecting the liquid composition to a cation exchange (CEX) chromatography as described herein. In some embodiments, the methods further comprise (ii) collecting eluate from the cation exchange (CEX) chromatography, thereby obtaining an rASM preparation with modulated specific activity. In some embodiments, the methods further comprise subjecting the eluate obtained from the CEX chromatography directly or indirectly to an IMAC. In some embodiments, the methods further comprise collecting eluate from the IMAC thereby obtaining an rASM preparation with further modulated specific activity. Optionally, the sequence of the CEX chromatography and IMAC can be exchanged. For example, in some embodiments, the methods comprise (i) subjecting the liquid composition to an IMAC as described herein. In some embodiments, the methods further comprise (ii) collecting eluate from the IMAC, thereby obtaining an rASM preparation with modulated specific activity. In some embodiments, the methods further comprise subjecting the eluate obtained from the IMAC directly or indirectly to a CEX chromatography. In some embodiments, the methods further comprise collecting eluate from the CEX chromatography thereby obtaining an rASM preparation with further modulated specific activity. In certain embodiments, the obtained rASM preparation has a reduced specific activity. In some embodiments, specific activity in the obtained rASM preparation is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more. In some embodiments, specific activity in the obtained rASM preparation is about 5-50 U/mg, such as about 10-40 U/mg, about 15-45 U/mg, about 10-30 U/mg, about 15-35 U/mg, or about 10-20 U/mg. In some embodiments, specific activity in the obtained rASM preparation is about 5 U/mg, about 10 U/mg, about 15 U/mg, about 20 U/mg, about 25 U/mg, about 30 U/mg, about 35 U/mg, about 40 U/mg, about 45 U/mg, about 50 U/mg.

In some embodiments, rASM of the present disclosure can be produced in host cells. For example, host cells expressing rASM and/or cell culture comprising rASM are collected to produce a harvest. In methods described herein, the harvested cells and/or cell culture may be used as is, as appropriate, or concentrated. In some embodiments, the harvest is concentrated. In some embodiments, the harvest is clarified by a suitable method (e.g., filtration) before being purified to produce a clarified harvest. In some embodiments, the harvest is lysed to produce a lysate.

In some embodiments, a cation exchange (CEX) chromatography is used in the process to reduce host cell proteins, and/or to provide viral clearance. The terms “CEX,” “cation exchange media,” “cation exchange resin,” and “cation exchange material,” as used herein, refer to a solid phase which is negatively charged, and which thus has free cations for exchange with cations in an aqueous solution passed over or through the solid phase. A negatively charged ligand attached to the solid phase to form the cation exchange resin may, e.g., be a carboxylate or sulfonate. Commercially available cation exchange resins include, but are not limited to, carboxy-methyl-cellulose, sulphopropyl (SP) immobilized on agarose (e.g., SP-SEPHAROSE FAST FLOW™ or SP-SEPHAROSE HIGH PERFORMANCE™ from Pharmacia) and sulphonyl immobilized on agarose (e.g., S-SEPHAROSE FAST FLOW™ from Pharmacia). In some embodiments, the cation exchange chromatography comprising a resin selected from the group consisting of carboxymethyl (CM), sulfoethyl (SE), sulfopropyl (SP), phosphate (P) and sulfonate (S). In some embodiments, the CEX chromatography comprises (1) loading a liquid composition comprising rASM to a CEX chromatography membrane or column; (2) washing the CEX chromatography membrane or column with a wash buffer; (3) eluting rASM from the CEX chromatography membrane or column with an elution buffer; and (4) collecting eluate comprising rASM. The term “equilibration buffer” refers to a buffer used to equilibrate the chromatography resin prior to loading a sample to the chromatography. The term “wash buffer” refers to a buffer used to wash the chromatography resin after the sample is loaded onto the chromatography. In some embodiments, the wash buffer and the equilibration buffer are the same or different. In some cases, the wash buffer and the loading buffer may be the same. “Washing” a chromatography media is meant to encompass passing an appropriate buffer through or over the media after a sample is loaded to the chromatography media. An “elution buffer” is used to elute the target protein from the solid phase. The conductivity and/or pH of the elution buffer is/are usually such that the target protein is eluted from the chromatography resin. To “elute” a molecule (e.g., a polypeptide of interest or an impurity) from a chromatography resin is meant to remove the molecule therefrom by altering the solution conditions such that the buffer competes with the molecule of interest for binding to the chromatography resin, or such that the binding interaction between the molecule of interest and the resin is weakened, causing the molecule of interest to dissociate. A non-limiting example is to elute a molecule from an ion exchange resin by altering the ionic strength of the buffer surrounding the ion exchange material such that the buffer competes with the molecule for the charged sites on the ion exchange material. The term “eluate,” as used herein, refers to a solution containing a molecule of interest obtained via elution as well as the flow-through fraction containing the target protein of interest obtained as a result of flow-through purification. In some embodiments, the term “eluate” refers to the elution pool from a bind and elute chromatography step.

In some embodiments, the CEX chromatography comprises (1) loading a composition comprising rASM to a CEX chromatography membrane or a CEX chromatography column. In some embodiments, the composition comprises an unmodified rASM isoform and at least one modified rASM isoform as described herein.

In some embodiments, the CEX chromatography further comprises (2) washing the membrane or the column with a wash buffer having a first optimal pH and a first optimal salt concentration. The first optimal pH and the first optimal salt concentration are predetermined depending on the resin and starting specific activity of the composition. In some embodiments, the wash buffer comprises a pH buffering system based on a phosphate salt, such as sodium phosphate. In some embodiments, the sodium phosphate concentration is about 5 to 100 mM, such as about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM.

In some embodiments, the wash buffer comprises a salt at an optimal salt concentration. In some embodiments, the salt is sodium chloride. In some embodiments, the sodium chloride concentration is about 5 to 100 mM, such as about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM.

In some embodiments, the first optimal salt concentration and the first optimal pH is selected from the conditions in Table 2a below.

TABLE 2a
Salt (NaCl)/pH conditions for CEX wash buffer

| | pH 6.1 | pH 6.2 | pH 6.3 | pH 6.4 | pH 6.5 | pH 6.6 | pH 6.7 | pH 6.8 | pH 6.9 | pH 7.0 |
|---|---|---|---|---|---|---|---|---|---|---|
| 0 | CEX | CEX | CEX | CEX | CEX | CEX | CEX | CEX | CEX | CEX |
| mM | W1 | W26 | W51 | W76 | W101 | W126 | W151 | W176 | W201 | W226 |
| 2 | CEX | CEX | CEX | CEX | CEX | CEX | CEX | CEX | CEX | CEX |
| mM | W2 | W27 | W52 | W77 | W102 | W127 | W152 | W177 | W202 | W227 |
| 4 | CEX | CEX | CEX | CEX | CEX | CEX | CEX | CEX | CEX | CEX |
| mM | W3 | W28 | W53 | W78 | W103 | W128 | W153 | W178 | W203 | W228 |
| 6 | CEX | CEX | CEX | CEX | CEX | CEX | CEX | CEX | CEX | CEX |
| mM | W4 | W29 | W54 | W79 | W104 | W129 | W154 | W179 | W204 | W229 |
| 8 | CEX | CEX | CEX | CEX | CEX | CEX | CEX | CEX | CEX | CEX |
| mM | W5 | W30 | W55 | W80 | W105 | W130 | W155 | W180 | W205 | W230 |
| 10 | CEX | CEX | CEX | CEX | CEX | CEX | CEX | CEX | CEX | CEX |
| mM | W6 | W31 | W56 | W81 | W106 | W131 | W156 | W181 | W206 | W231 |
| 12 | CEX | CEX | CEX | CEX | CEX | CEX | CEX | CEX | CEX | CEX |
| mM | W7 | W32 | W57 | W82 | W107 | W132 | W157 | W182 | W207 | W232 |
| 14 | CEX | CEX | CEX | CEX | CEX | CEX | CEX | CEX | CEX | CEX |
| mM | W8 | W33 | W58 | W83 | W108 | W133 | W158 | W183 | W208 | W233 |
| 16 | CEX | CEX | CEX | CEX | CEX | CEX | CEX | CEX | CEX | CEX |
| mM | W9 | W34 | W59 | W84 | W109 | W134 | W159 | W184 | W209 | W234 |
| 18 | CEX | CEX | CEX | CEX | CEX | CEX | CEX | CEX | CEX | CEX |
| mM | W10 | W35 | W60 | W85 | W110 | W135 | W160 | W185 | W210 | W235 |
| 20 | CEX | CEX | CEX | CEX | CEX | CEX | CEX | CEX | CEX | CEX |
| mM | W11 | W36 | W61 | W86 | W111 | W136 | W161 | W186 | W211 | W236 |
| 22 | CEX | CEX | CEX | CEX | CEX | CEX | CEX | CEX | CEX | CEX |
| mM | W12 | W37 | W62 | W87 | W112 | W137 | W162 | W187 | W212 | W237 |
| 25 | CEX | CEX | CEX | CEX | CEX | CEX | CEX | CEX | CEX | CEX |
| mM | W13 | W38 | W63 | W88 | W113 | W138 | W163 | W188 | W213 | W238 |
| 30 | CEX | CEX | CEX | CEX | CEX | CEX | CEX | CEX | CEX | CEX |
| mM | W14 | W39 | W64 | W89 | W114 | W139 | W164 | W189 | W214 | W239 |
| 35 | CEX | CEX | CEX | CEX | CEX | CEX | CEX | CEX | CEX | CEX |
| mM | W15 | W40 | W65 | W90 | W115 | W140 | W165 | W190 | W215 | W240 |
| 40 | CEX | CEX | CEX | CEX | CEX | CEX | CEX | CEX | CEX | CEX |
| mM | W16 | W41 | W66 | W91 | W116 | W141 | W166 | W191 | W216 | W241 |
| 45 | CEX | CEX | CEX | CEX | CEX | CEX | CEX | CEX | CEX | CEX |
| mM | W17 | W42 | W67 | W92 | W117 | W142 | W167 | W192 | W217 | W242 |
| 50 | CEX | CEX | CEX | CEX | CEX | CEX | CEX | CEX | CEX | CEX |
| mM | W18 | W43 | W68 | W93 | W118 | W143 | W168 | W193 | W218 | W243 |
| 55 | CEX | CEX | CEX | CEX | CEX | CEX | CEX | CEX | CEX | CEX |
| mM | W19 | W44 | W69 | W94 | W119 | W144 | W169 | W194 | W219 | W244 |
| 60 | CEX | CEX | CEX | CEX | CEX | CEX | CEX | CEX | CEX | CEX |
| mM | W20 | W45 | W70 | W95 | W120 | W145 | W170 | W195 | W220 | W245 |
| 65 | CEX | CEX | CEX | CEX | CEX | CEX | CEX | CEX | CEX | CEX |
| mM | W21 | W46 | W71 | W96 | W121 | W146 | W171 | W196 | W221 | W246 |
| 70 | CEX | CEX | CEX | CEX | CEX | CEX | CEX | CEX | CEX | CEX |
| mM | W22 | W47 | W72 | W97 | W122 | W147 | W172 | W197 | W222 | W247 |
| 75 | CEX | CEX | CEX | CEX | CEX | CEX | CEX | CEX | CEX | CEX |
| mM | W23 | W48 | W73 | W98 | W123 | W148 | W173 | W198 | W223 | W248 |
| 80 | CEX | CEX | CEX | CEX | CEX | CEX | CEX | CEX | CEX | CEX |
| mM | W24 | W49 | W74 | W99 | W124 | W149 | W174 | W199 | W224 | W249 |
| 85 | CEX | CEX | CEX | CEX | CEX | CEX | CEX | CEX | CEX | CEX |
| mM | W25 | W50 | W75 | W100 | W125 | W150 | W175 | W200 | W225 | W250 |

Generally speaking, higher pH and/or higher salt concentration in the wash buffer leads to higher purity (e.g., higher HCP clearance), but lower specific activity and lower recovery rate. Accordingly, a CEX wash condition can be selected from CEX W1 to CEX W250 depending on the target purity and specific activity. In some embodiments, the condition is selected from CEX W1 to CEX W25; in some embodiments, the condition is selected from CEX W26 to CEX W50; in some embodiments, the condition is selected from CEX W51 to CEX W75; in some embodiments, the condition is selected from CEX W76 to CEX W100; in some embodiments, the condition is selected from CEX W101 to CEX W125; in some embodiments, the condition is selected from CEX W126 to CEX W150; in some embodiments, the condition is selected from CEX W151 to CEX W175; in some embodiments, the condition is selected from CEX W176 to CEX W200; in some embodiments, the condition is selected from CEX W201 to CEX W225; in some embodiments, the condition is selected from CEX W226 to CEX W250. In some embodiments, the condition is selected from CEX W36, CEX W61, CEX W86, CEX W111, CEX W37, CEX W62, CEX W87, CEX W112, CEX W38, CEX W63, CEX W88, CEX W113, CEX W39, CEX W64, CEX W89, CEX W114, CEX W40, CEX W65, CEX W90, and CEX W115. In some embodiments, the condition is selected from CEX W66, CEX W91, CEX 116, CEX W67, CEX W92, CEX W117, CEX W68, CEX W93, CEX W118, CEX W69, CEX W94, CEX W119, CEX W70, CEX W95, and CEX W120. In some embodiments, the condition is selected from CEX W141, CEX W166, CEX W191, CEX W142, CEX W167, CEX W192, CEX W143, CEX W168, CEX W193, CEX W144, CEX W169, CEX W194, CEX W145, CEX W170, and CEX W195. In some embodiments, the condition is selected from CEX W136, CEX W161, CEX W186, CEX W137, CEX W162, CEX W187, CEX W138, CEX W163, CEX W188, CEX W139, CEX W164, CEX W189, CEX W140, CEX W165, and CEX W190.

In some embodiments, the CEX chromatography further comprises (3) eluting the membrane or the column with an elution buffer having a second optimal pH and a second optimal salt concentration. In some embodiments, the second optimal pH is as the same as or close to the first optimal pH used in the wash buffer. In some embodiments, the second optimal salt concentration is predetermined depending on the first optimal salt concentration in the wash buffer. In general, a salt concentration higher than the first optimal salt concentration is used to elute rASM binding to the membrane or column. In some embodiments, the salt concentration in the elution buffer is about 150 mM, about 160 mM, about 170 mM, about 180 mM, about 190 mM, about 200 mM, about 210 mM, about 220 mM, about 230 mM, about 240 mM, or about 250 mM. In some embodiments, the elution buffer comprises a pH buffering system based on a phosphate salt, such as sodium phosphate. In some embodiments, the sodium phosphate concentration is about 5 to 100 mM, such as about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM. In some embodiments, the sodium phosphate concentration is the same as or similar to that in the CEX wash buffer.

In some embodiments, the elution buffer comprises a salt at an optimal salt concentration. In some embodiments, the salt is sodium chloride.

In some embodiments, the second optimal salt concentration and the second optimal pH of the elution buffer is selected from the conditions in Table 2b below.

TABLE 2b
Salt (NaCl)/pH conditions for CEX elution buffer

| | pH 6.1 | pH 6.2 | pH 6.3 | pH 6.4 | pH 6.5 | pH 6.6 | pH 6.7 | pH 6.8 | pH 6.9 | pH 7.0 |
|---|---|---|---|---|---|---|---|---|---|---|
| 110 | CEX | CEX | CEX | CEX | CEX | CEX | CEX | CEX | CEX | CEX |
| mM | E1 | E26 | E51 | E76 | E101 | E126 | E151 | E176 | E201 | E226 |
| 115 | CEX | CEX | CEX | CEX | CEX | CEX | CEX | CEX | CEX | CEX |
| mM | E2 | E27 | E52 | E77 | E102 | E127 | E152 | E177 | E202 | E227 |
| 120 | CEX | CEX | CEX | CEX | CEX | CEX | CEX | CEX | CEX | CEX |
| mM | E3 | E28 | E53 | E78 | E103 | E128 | E153 | E178 | E203 | E228 |
| 125 | CEX | CEX | CEX | CEX | CEX | CEX | CEX | CEX | CEX | CEX |
| mM | E4 | E29 | E54 | E79 | E104 | E129 | E154 | E179 | E204 | E229 |
| 130 | CEX | CEX | CEX | CEX | CEX | CEX | CEX | CEX | CEX | CEX |
| mM | E5 | E30 | E55 | E80 | E105 | E130 | E155 | E180 | E205 | E230 |
| 135 | CEX | CEX | CEX | CEX | CEX | CEX | CEX | CEX | CEX | CEX |
| mM | E6 | E31 | E56 | E81 | E106 | E131 | E156 | E181 | E206 | E231 |
| 140 | CEX | CEX | CEX | CEX | CEX | CEX | CEX | CEX | CEX | CEX |
| mM | E7 | E32 | E57 | E82 | E107 | E132 | E157 | E182 | E207 | E232 |
| 145 | CEX | CEX | CEX | CEX | CEX | CEX | CEX | CEX | CEX | CEX |
| mM | E8 | E33 | E58 | E83 | E108 | E133 | E158 | E183 | E208 | E233 |
| 150 | CEX | CEX | CEX | CEX | CEX | CEX | CEX | CEX | CEX | CEX |
| mM | E9 | E34 | E59 | E84 | E109 | E134 | E159 | E184 | E209 | E234 |
| 155 | CEX | CEX | CEX | CEX | CEX | CEX | CEX | CEX | CEX | CEX |
| mM | E10 | E35 | E60 | E85 | E110 | E135 | E160 | E185 | E210 | E235 |
| 160 | CEX | CEX | CEX | CEX | CEX | CEX | CEX | CEX | CEX | CEX |
| mM | E11 | E36 | E61 | E86 | E111 | E136 | E161 | E186 | E211 | E236 |
| 165 | CEX | CEX | CEX | CEX | CEX | CEX | CEX | CEX | CEX | CEX |
| mM | E12 | E37 | E62 | E87 | E112 | E137 | E162 | E187 | E212 | E237 |
| 170 | CEX | CEX | CEX | CEX | CEX | CEX | CEX | CEX | CEX | CEX |
| mM | E13 | E38 | E63 | E88 | E113 | E138 | E163 | E188 | E213 | E238 |
| 175 | CEX | CEX | CEX | CEX | CEX | CEX | CEX | CEX | CEX | CEX |
| mM | E14 | E39 | E64 | E89 | E114 | E139 | E164 | E189 | E214 | E239 |
| 180 | CEX | CEX | CEX | CEX | CEX | CEX | CEX | CEX | CEX | CEX |
| mM | E15 | E40 | E65 | E90 | E115 | E140 | E165 | E190 | E215 | E240 |
| 185 | CEX | CEX | CEX | CEX | CEX | CEX | CEX | CEX | CEX | CEX |
| mM | E16 | E41 | E66 | E91 | E116 | E141 | E166 | E191 | E216 | E241 |
| 190 | CEX | CEX | CEX | CEX | CEX | CEX | CEX | CEX | CEX | CEX |
| mM | E17 | E42 | E67 | E92 | E117 | E142 | E167 | E192 | E217 | E242 |
| 195 | CEX | CEX | CEX | CEX | CEX | CEX | CEX | CEX | CEX | CEX |
| mM | E18 | E43 | E68 | E93 | E118 | E143 | E168 | E193 | E218 | E243 |
| 200 | CEX | CEX | CEX | CEX | CEX | CEX | CEX | CEX | CEX | CEX |
| mM | E19 | E44 | E69 | E94 | E119 | E144 | E169 | E194 | E219 | E244 |
| 205 | CEX | CEX | CEX | CEX | CEX | CEX | CEX | CEX | CEX | CEX |
| mM | E20 | E45 | E70 | E95 | E120 | E145 | E170 | E195 | E220 | E245 |
| 210 | CEX | CEX | CEX | CEX | CEX | CEX | CEX | CEX | CEX | CEX |
| mM | E21 | E46 | E71 | E96 | E121 | E146 | E171 | E196 | E221 | E246 |
| 215 | CEX | CEX | CEX | CEX | CEX | CEX | CEX | CEX | CEX | CEX |
| mM | E22 | E47 | E72 | E97 | E122 | E147 | E172 | E197 | E222 | E247 |
| 220 | CEX | CEX | CEX | CEX | CEX | CEX | CEX | CEX | CEX | CEX |
| mM | E23 | E48 | E73 | E98 | E123 | E148 | E173 | E198 | E223 | E248 |
| 225 | CEX | CEX | CEX | CEX | CEX | CEX | CEX | CEX | CEX | CEX |
| mM | E24 | E49 | E74 | E99 | E124 | E149 | E174 | E199 | E224 | E249 |
| 230 | CEX | CEX | CEX | CEX | CEX | CEX | CEX | CEX | CEX | CEX |
| mM | E25 | E50 | E75 | E100 | E125 | E150 | E175 | E200 | E225 | E250 |

In some embodiments, the condition is selected from CEX E1 to CEX E25; in some embodiments, the condition is selected from CEX E26 to CEX E50; in some embodiments, the condition is selected from CEX E51 to CEX E75; in some embodiments, the condition is selected from CEX E76 to CEX E100; in some embodiments, the condition is selected from CEX E101 to CEX E125; in some embodiments, the condition is selected from CEX E126 to CEX E150; in some embodiments, the condition is selected from CEX E151 to CEX E175; in some embodiments, the condition is selected from CEX E176 to CEX E200; in some embodiments, the condition is selected from CEX E201 to CEX E225; in some embodiments, the condition is selected from CEX E226 to CEX E250. In some embodiments, the condition is selected from CEX E36, CEX E61, CEX E86, CEX E111, CEX E37, CEX E62, CEX E87, CEX E112, CEX E38, CEX E63, CEX E88, CEX E113, CEX E39, CEX E64, CEX E89, CEX E114, CEX E40, CEX E65, CEX E90, and CEX E115. In some embodiments, the condition is selected from CEX E66, CEX E91, CEX 116, CEX E67, CEX E92, CEX E117, CEX E68, CEX E93, CEX E118, CEX E69, CEX E94, CEX E119, CEX E70, CEX E95, and CEX E120. In some embodiments, the condition is selected from CEX E141, CEX E166, CEX E191, CEX E142, CEX E167, CEX E192, CEX E143, CEX E168, CEX E193, CEX E144, CEX E169, CEX E194, CEX E145, CEX E170, and CEX E195. In some embodiments, the condition is selected from CEX E136, CEX E161, CEX E186, CEX E137, CEX E162, CEX E187, CEX E138, CEX E163, CEX E188, CEX E139, CEX E164, CEX E189, CEX E140, CEX E165, and CEX E190.

In some embodiments, the CEX chromatography comprises any one of the following washing/elution condition combinations:
(1) CEX washing condition of any one of CEX W1 to CEX W25, and CEX elution condition of any one of CEX E1 to CEX E25;
(2) CEX washing condition of any one of CEX W1 to CEX W25, and CEX elution condition of any one of CEX E101 to CEX E125;
(3) CEX washing condition of any one of CEX W1 to CEX W25, and CEX elution condition of any one of CEX E226 to CEX E250;
(4) CEX washing condition of any one of CEX W101 to CEX W125, and CEX elution condition of any one of CEX E1 to CEX E25;
(5) CEX washing condition of any one of CEX W101 to CEX W125, and CEX elution condition of any one of CEX E101 to CEX E125;
(6) CEX washing condition of any one of CEX W101 to CEX W125, and CEX elution condition of any one of CEX E226 to CEX E250;
(7) CEX washing condition of any one of CEX W226 to CEX W250, and CEX elution condition of any one of CEX E1 to CEX E25;
(8) CEX washing condition of any one of CEX W226 to CEX W250, and CEX elution condition of any one of CEX E101 to CEX E125;
(9) CEX washing condition of any one of CEX W226 to CEX W250, and CEX elution condition of any one of CEX E226 to CEX E250.

The CEX chromatography step as described herein may be either performed under refrigerated condition (e.g., 8±3° C.), or under ambient temperature.

In some embodiments, methods of the present disclosure further comprise an Immobilized Metal Affinity Chromatography (IMAC) to reduce host cell proteins. The term “IMAC,” as used herein, refers to a solid phase which is based on the affinity of transition metal ions such as Zn²⁺, Cu²⁺, Ni²⁺, and Co²⁺ to histidine or cysteine in aqueous solutions. Types of IMACs are described in Block et al., Methods in Enzymology (2009) 463:439-73. In further embodiments, the IMAC resin is charged with a divalent ion. In yet further embodiments, the divalent metal ion is nickel, copper, cobalt, or zinc. In more specific embodiments, the divalent metal ion is zinc.

In some embodiments, the IMAC chromatography comprises (1) loading a liquid composition comprising rASM to an IMAC chromatography membrane or column; (2) washing the IMAC chromatography membrane or column with a wash buffer; (3) eluting rASM from the IMAC chromatography membrane or column with an elution buffer; and (4) collecting the eluate comprising rASM.

In some embodiments, the eluate obtained in the CEX chromatography is subjected to an IMAC chromatography in a bind-and-elute mode.

In some embodiments, the IMAC comprises (1) loading a composition comprising rASM to an IMAC chromatography membrane or an IMAC chromatography column. In some embodiments, the composition comprises an unmodified rASM isoform and at least one modified rASM isoform as described herein.

In some embodiments, the IMAC chromatography further comprises (2) washing the membrane or the column with a wash buffer having a third optimal pH and a third optimal salt concentration. The third optimal pH and the third optimal salt concentration are predetermined depending on the resin and starting specific activity of the composition. In some embodiments, the wash buffer comprises a pH buffering system based on a phosphate salt, such as sodium phosphate. In some embodiments, the sodium phosphate concentration is about 1 to 100 mM, such as about 1 mM, about 2 mM, about 3 mM, about 4 mM, about 5 mM, about 6 mM, about 7 mM, about 8 mM, about 9 mM, about 17 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM. In some embodiments, the third optimal salt concentration and the third optimal pH is selected from the conditions in Table 3 below.

TABLE 3
Salt (NaCl)/pH conditions for IMAC wash buffer

| | pH 5.8 | pH 6.0 | pH 6.2 | pH 6.4 | pH 6.6 | pH 6.8 | pH 6.9 | pH 7.0 | pH 7.1 | pH 7.2 |
|---|---|---|---|---|---|---|---|---|---|---|
| 0 | IMAC | IMAC | IMAC | IMAC | IMAC | IMAC | IMAC | IMAC | IMAC | IMAC |
| mM | W1 | W26 | W51 | W76 | W101 | W126 | W151 | W176 | W201 | W226 |
| 2 | IMAC | IMAC | IMAC | IMAC | IMAC | IMAC | IMAC | IMAC | IMAC | IMAC |
| mM | W2 | W27 | W52 | W77 | W102 | W127 | W152 | W177 | W202 | W227 |
| 4 | IMAC | IMAC | IMAC | IMAC | IMAC | IMAC | IMAC | IMAC | IMAC | IMAC |
| mM | W3 | W28 | W53 | W78 | W103 | W128 | W153 | W178 | W203 | W228 |
| 6 | IMAC | IMAC | IMAC | IMAC | IMAC | IMAC | IMAC | IMAC | IMAC | IMAC |
| mM | W4 | W29 | W54 | W79 | W104 | W129 | W154 | W179 | W204 | W229 |
| 8 | IMAC | IMAC | IMAC | IMAC | IMAC | IMAC | IMAC | IMAC | IMAC | IMAC |
| mM | W5 | W30 | W55 | W80 | W105 | W130 | W155 | W180 | W205 | W230 |
| 10 | IMAC | IMAC | IMAC | IMAC | IMAC | IMAC | IMAC | IMAC | IMAC | IMAC |
| mM | W6 | W31 | W56 | W81 | W106 | W131 | W156 | W181 | W206 | W231 |
| 12 | IMAC | IMAC | IMAC | IMAC | IMAC | IMAC | IMAC | IMAC | IMAC | IMAC |
| mM | W7 | W32 | W57 | W82 | W107 | W132 | W157 | W182 | W207 | W232 |
| 14 | IMAC | IMAC | IMAC | IMAC | IMAC | IMAC | IMAC | IMAC | IMAC | IMAC |
| mM | W8 | W33 | W58 | W83 | W108 | W133 | W158 | W183 | W208 | W233 |
| 16 | IMAC | IMAC | IMAC | IMAC | IMAC | IMAC | IMAC | IMAC | IMAC | IMAC |
| mM | W9 | W34 | W59 | W84 | W109 | W134 | W159 | W184 | W209 | W234 |
| 18 | IMAC | IMAC | IMAC | IMAC | IMAC | IMAC | IMAC | IMAC | IMAC | IMAC |
| mM | W10 | W35 | W60 | W85 | W110 | W135 | W160 | W185 | W210 | W235 |
| 20 | IMAC | IMAC | IMAC | IMAC | IMAC | IMAC | IMAC | IMAC | IMAC | IMAC |
| mM | W11 | W36 | W61 | W86 | W111 | W136 | W161 | W186 | W211 | W236 |
| 25 | IMAC | IMAC | IMAC | IMAC | IMAC | IMAC | IMAC | IMAC | IMAC | IMAC |
| mM | W12 | W37 | W62 | W87 | W112 | W137 | W162 | W187 | W212 | W237 |
| 30 | IMAC | IMAC | IMAC | IMAC | IMAC | IMAC | IMAC | IMAC | IMAC | IMAC |
| mM | W13 | W38 | W63 | W88 | W113 | W138 | W163 | W188 | W213 | W238 |
| 35 | IMAC | IMAC | IMAC | IMAC | IMAC | IMAC | IMAC | IMAC | IMAC | IMAC |
| mM | W14 | W39 | W64 | W89 | W114 | W139 | W164 | W189 | W214 | W239 |
| 40 | IMAC | IMAC | IMAC | IMAC | IMAC | IMAC | IMAC | IMAC | IMAC | IMAC |
| mM | W15 | W40 | W65 | W90 | W115 | W140 | W165 | W190 | W215 | W240 |
| 45 | IMAC | IMAC | IMAC | IMAC | IMAC | IMAC | IMAC | IMAC | IMAC | IMAC |
| mM | W16 | W41 | W66 | W91 | W116 | W141 | W166 | W191 | W216 | W241 |
| 50 | IMAC | IMAC | IMAC | IMAC | IMAC | IMAC | IMAC | IMAC | IMAC | IMAC |
| mM | W17 | W42 | W67 | W92 | W117 | W142 | W167 | W192 | W217 | W242 |
| 55 | IMAC | IMAC | IMAC | IMAC | IMAC | IMAC | IMAC | IMAC | IMAC | IMAC |
| mM | W18 | W43 | W68 | W93 | W118 | W143 | W168 | W193 | W218 | W243 |
| 60 | IMAC | IMAC | IMAC | IMAC | IMAC | IMAC | IMAC | IMAC | IMAC | IMAC |
| mM | W19 | W44 | W69 | W94 | W119 | W144 | W169 | W194 | W219 | W244 |
| 65 | IMAC | IMAC | IMAC | IMAC | IMAC | IMAC | IMAC | IMAC | IMAC | IMAC |
| mM | W20 | W45 | W70 | W95 | W120 | W145 | W170 | W195 | W220 | W245 |
| 70 | IMAC | IMAC | IMAC | IMAC | IMAC | IMAC | IMAC | IMAC | IMAC | IMAC |
| mM | W21 | W46 | W71 | W96 | W121 | W146 | W171 | W196 | W221 | W246 |
| 75 | IMAC | IMAC | IMAC | IMAC | IMAC | IMAC | IMAC | IMAC | IMAC | IMAC |
| mM | W22 | W47 | W72 | W97 | W122 | W147 | W172 | W197 | W222 | W247 |
| 80 | IMAC | IMAC | IMAC | IMAC | IMAC | IMAC | IMAC | IMAC | IMAC | IMAC |
| mM | W23 | W48 | W73 | W98 | W123 | W148 | W173 | W198 | W223 | W248 |
| 85 | IMAC | IMAC | IMAC | IMAC | IMAC | IMAC | IMAC | IMAC | IMAC | IMAC |
| mM | W24 | W49 | W74 | W99 | W124 | W149 | W174 | W199 | W224 | W249 |
| 90 | IMAC | IMAC | IMAC | IMAC | IMAC | IMAC | IMAC | IMAC | IMAC | IMAC |
| mM | W25 | W50 | W75 | W100 | W125 | W150 | W175 | W200 | W225 | W250 |

Generally speaking, lower pH and/or higher salt concentration in the IMAC wash buffer leads to higher purity (e.g., higher HCP clearance), but lower specific activity and lower recovery rate. Accordingly, an IMAC wash condition can be selected from IMAC W1 to IMAC W250 depending on the target purity and specific activity. In some embodiments, the condition is selected from IMAC W1 to IMAC W25; in some embodiments, the condition is selected from IMAC W26 to IMAC W50; in some embodiments, the condition is selected from IMAC W51 to IMAC W75; in some embodiments, the condition is selected from IMAC W76 to IMAC W100; in some embodiments, the condition is selected from IMAC W101 to IMAC W125; in some embodiments, the condition is selected from IMAC W126 to IMAC W150; in some embodiments, the condition is selected from IMAC W151 to IMAC W175; in some embodiments, the condition is selected from IMAC W176 to IMAC W200; in some embodiments, the condition is selected from IMAC W201 to IMAC W225; in some embodiments, the condition is selected from IMAC W226 to IMAC W250. In some embodiments, the condition is selected from IMAC W1, IMAC W26, IMAC W51, IMAC W76, IMAC W101, IMAC W2, IMAC W27, IMAC W52, IMAC W77, IMAC W102, IMAC W3, IMAC W28, IMAC W53, IMAC W78, IMAC W103, IMAC W4, IMAC W29, IMAC W54, IMAC W79, IMAC W104, IMAC W5, IMAC W30, IMAC W55, IMAC W80, IMAC W105. In some embodiments, the condition is selected from IMAC W126, IMAC, W151, IMAC W176, IMAC W201, IMAC W226, IMAC W127, IMAC W152, IMAC W177, IMAC W202, IMAC W227, IMAC W128, IMAC W153, IMAC W178, IMAC W203, IMAC W228, IMAC W129, IMAC W154, IMAC W179, IMAC W204, IMAC W229, IMAC W130, IMAC W155, IMAC W180, IMAC W205, and IMAC W230. In some embodiments, the condition is selected from IMAC W6, IMAC W31, IMAC W56, IMAC W81, IMAC W106, IMAC W7, IMAC W32, IMAC W57, IMAC W82, IMAC W107, IMAC W8, IMAC W33, IMAC W58, IMAC W83, IMAC W108, IMAC W9, IMAC W34, IMAC W59, IMAC W84, IMAC W109, IMAC W10, IMAC W35, IMAC W60, IMAC W85, and IMAC W110. In some embodiments, the condition is selected from IMAC W131, IMAC W156, IMAC W181, IMAC W206, IMAC W231, IMAC W132, IMAC W157, IMAC W182, IMAC W207, IMAC W232, IMAC W133, IMAC W158, IMAC W183, IMAC W208, IMAC W233, IMAC W134, IMAC W159, IMAC W184, IMAC W209, IMAC W234, IMAC W135, IMAC W160, IMAC W185, IMAC W210, and IMAC W235.

Optionally, the IMAC chromatography comprises a further wash step (IMAC wash 2). In some embodiments, the second wash step comprises using a pH lower than that used in the first IMAC wash step. In some embodiments, the second wash step comprises using a pH about 0.1, about 0.2, about 0.3, about 0.4, about 0.5, about 0.6, about 0.7, about 0.8, about 0.9, about 1.0, or about 1.2 lower than that used in the first IMAC wash step. In some embodiments, the second wash step comprises using a salt condition the same or close to that used in the first IMAC wash step. In some embodiments, the second wash step comprises a higher salt concentration (e.g., NaCl) than the first wash. In some embodiments, the second wash step comprises a sodium chloride concentration of at least 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 120 mM, 130 mM, 140 mM, 150 mM, or more.

In some embodiments, the second wash step comprises using a wash buffer with salt concentration that is the same or close to that used in the first IMAC wash step.

In some embodiments, the IMAC chromatography further comprises (3) eluting the membrane or the column with an elution buffer having a fourth optimal pH and a fourth optimal salt concentration. In some embodiments, the elution buffer comprises a pH buffering system based on a phosphate salt, such as sodium phosphate. In some embodiments, the sodium phosphate concentration is about 5 to 100 mM, such as about 5 mM, about 10 mM, about 15 mM, about 20 mM, about 25 mM, about 30 mM, about 35 mM, about 40 mM, about 45 mM, about 50 mM, about 60 mM, about 70 mM, about 80 mM, about 90 mM, or about 100 mM. In some embodiments, the sodium phosphate concentration is the same as or similar to that in the IMAC wash buffer. In some embodiments, the fourth optimal salt concentration and the fourth optimal pH are predetermined depending on the third optimal salt concentration and third optimal pH in the IMAC wash buffer.

In some embodiments, the fourth optimal salt concentration and the fourth optimal pH is selected from the conditions in Table 4 below.

TABLE 4
Salt (NaCl)/pH conditions for IMAC elution buffer

| | pH 6.3 | pH 6.4 | pH 6.5 | pH 6.6 | pH 6.7 | pH 6.8 | pH 6.9 | pH 7.0 | pH 7.1 | pH 7.2 |
|---|---|---|---|---|---|---|---|---|---|---|
| 0.1M | IMAC | IMAC | IMAC | IMAC | IMAC | IMAC | IMAC | IMAC | IMAC | IMAC |
| | E1 | E26 | E51 | E76 | E101 | E126 | E151 | E176 | E201 | E226 |
| 0.2M | IMAC | IMAC | IMAC | IMAC | IMAC | IMAC | IMAC | IMAC | IMAC | IMAC |
| | E2 | E27 | E52 | E77 | E102 | E127 | E152 | E177 | E202 | E227 |
| 0.3M | IMAC | IMAC | IMAC | IMAC | IMAC | IMAC | IMAC | IMAC | IMAC | IMAC |
| | E3 | E28 | E53 | E78 | E103 | E128 | E153 | E178 | E203 | E228 |
| 0.4M | IMAC | IMAC | IMAC | IMAC | IMAC | IMAC | IMAC | IMAC | IMAC | IMAC |
| | E4 | E29 | E54 | E79 | E104 | E129 | E154 | E179 | E204 | E229 |
| 0.5M | IMAC | IMAC | IMAC | IMAC | IMAC | IMAC | IMAC | IMAC | IMAC | IMAC |
| | E5 | E30 | E55 | E80 | E105 | E130 | E155 | E180 | E205 | E230 |
| 0.6M | IMAC | IMAC | IMAC | IMAC | IMAC | IMAC | IMAC | IMAC | IMAC | IMAC |
| | E6 | E31 | E56 | E81 | E106 | E131 | E156 | E181 | E206 | E231 |
| 0.7M | IMAC | IMAC | IMAC | IMAC | IMAC | IMAC | IMAC | IMAC | IMAC | IMAC |
| | E7 | E32 | E57 | E82 | E107 | E132 | E157 | E182 | E207 | E232 |
| 0.8M | IMAC | IMAC | IMAC | IMAC | IMAC | IMAC | IMAC | IMAC | IMAC | IMAC |
| | E8 | E33 | E58 | E83 | E108 | E133 | E158 | E183 | E208 | E233 |
| 0.9M | IMAC | IMAC | IMAC | IMAC | IMAC | IMAC | IMAC | IMAC | IMAC | IMAC |
| | E9 | E34 | E59 | E84 | E109 | E134 | E159 | E184 | E209 | E234 |
| 1.0M | IMAC | IMAC | IMAC | IMAC | IMAC | IMAC | IMAC | IMAC | IMAC | IMAC |
| | E10 | E35 | E60 | E85 | E110 | E135 | E160 | E185 | E210 | E235 |
| 1.1M | IMAC | IMAC | IMAC | IMAC | IMAC | IMAC | IMAC | IMAC | IMAC | IMAC |
| | E11 | E36 | E61 | E86 | E111 | E136 | E161 | E186 | E211 | E236 |
| 1.2M | IMAC | IMAC | IMAC | IMAC | IMAC | IMAC | IMAC | IMAC | IMAC | IMAC |
| | E12 | E37 | E62 | E87 | E112 | E137 | E162 | E187 | E212 | E237 |
| 1.3M | IMAC | IMAC | IMAC | IMAC | IMAC | IMAC | IMAC | IMAC | IMAC | IMAC |
| | E13 | E38 | E63 | E88 | E113 | E138 | E163 | E188 | E213 | E238 |
| 1.4M | IMAC | IMAC | IMAC | IMAC | IMAC | IMAC | IMAC | IMAC | IMAC | IMAC |
| | E14 | E39 | E64 | E89 | E114 | E139 | E164 | E189 | E214 | E239 |
| 1.5M | IMAC | IMAC | IMAC | IMAC | IMAC | IMAC | IMAC | IMAC | IMAC | IMAC |
| | E15 | E40 | E65 | E90 | E115 | E140 | E165 | E190 | E215 | E240 |
| 1.6M | IMAC | IMAC | IMAC | IMAC | IMAC | IMAC | IMAC | IMAC | IMAC | IMAC |
| | E16 | E41 | E66 | E91 | E116 | E141 | E166 | E191 | E216 | E241 |
| 1.7M | IMAC | IMAC | IMAC | IMAC | IMAC | IMAC | IMAC | IMAC | IMAC | IMAC |
| | E17 | E42 | E67 | E92 | E117 | E142 | E167 | E192 | E217 | E242 |
| 1.8M | IMAC | IMAC | IMAC | IMAC | IMAC | IMAC | IMAC | IMAC | IMAC | IMAC |
| | E18 | E43 | E68 | E93 | E118 | E143 | E168 | E193 | E218 | E243 |
| 1.9M | IMAC | IMAC | IMAC | IMAC | IMAC | IMAC | IMAC | IMAC | IMAC | IMAC |
| | E19 | E44 | E69 | E94 | E119 | E144 | E169 | E194 | E219 | E244 |
| 2.0M | IMAC | IMAC | IMAC | IMAC | IMAC | IMAC | IMAC | IMAC | IMAC | IMAC |
| | E20 | E45 | E70 | E95 | E120 | E145 | E170 | E195 | E220 | E245 |
| 2.1M | IMAC | IMAC | IMAC | IMAC | IMAC | IMAC | IMAC | IMAC | IMAC | IMAC |
| | E21 | E46 | E71 | E96 | E121 | E146 | E171 | E196 | E221 | E246 |
| 2.2M | IMAC | IMAC | IMAC | IMAC | IMAC | IMAC | IMAC | IMAC | IMAC | IMAC |
| | E22 | E47 | E72 | E97 | E122 | E147 | E172 | E197 | E222 | E247 |
| 2.3M | IMAC | IMAC | IMAC | IMAC | IMAC | IMAC | IMAC | IMAC | IMAC | IMAC |
| | E23 | E48 | E73 | E98 | E123 | E148 | E173 | E198 | E223 | E248 |
| 2.4M | IMAC | IMAC | IMAC | IMAC | IMAC | IMAC | IMAC | IMAC | IMAC | IMAC |
| | E24 | E49 | E74 | E99 | E124 | E149 | E174 | E199 | E224 | E249 |
| 2.5M | IMAC | IMAC | IMAC | IMAC | IMAC | IMAC | IMAC | IMAC | IMAC | IMAC |
| | E25 | E50 | E75 | E100 | E125 | E150 | E175 | E200 | E225 | E250 |

An IMAC elution condition can be selected from IMAC E1 to IMAC E250 depending on the target purity and specific activity. In some embodiments, the condition is selected from IMAC E1 to IMAC E25; in some embodiments, the condition is selected from IMAC E26 to IMAC E50; in some embodiments, the condition is selected from IMAC E51 to IMAC E75; in some embodiments, the condition is selected from IMAC E76 to IMAC E100; in some embodiments, the condition is selected from IMAC E101 to IMAC E125; in some embodiments, the condition is selected from IMAC E126 to IMAC E150; in some embodiments, the condition is selected from IMAC E151 to IMAC E175; in some embodiments, the condition is selected from IMAC E176 to IMAC E200; in some embodiments, the condition is selected from IMAC E201 to IMAC E225; in some embodiments, the condition is selected from IMAC E226 to IMAC E250. In some embodiments, the condition is selected from IMAC E13, IMAC E38, IMAC E63, IMAC E88, IMAC E113, IMAC E14, IMAC E39, IMAC E64, IMAC E89, IMAC E114, IMAC E15, IMAC E40, IMAC E65, IMAC E90, IMAC E115, IMAC E16, IMAC E41, IMAC E66, IMAC E91, IMAC E116, IMAC E17, IMAC E42, IMAC E67, IMAC E92, IMAC E117, IMAC E18, IMAC E43, IMAC E68, IMAC E93, and IMAC E118. In some embodiments, the condition is selected from IMAC E138, IMAC E163, IMAC E188, IMAC E213, IMAC E238, IMAC E139, IMAC E164, IMAC E189, IMAC E214, IMAC E239, IMAC E140, IMAC E165, IMAC E190, IMAC E215, IMAC E240, IMAC E141, IMAC E166, IMAC E191, IMAC E216, IMAC E241, IMAC E142, IMAC E167, IMAC E192, IMAC E217, and IMAC E242. In some embodiments, the condition is selected from IMAC E19, IMAC E44, IMAC E69, IMAC E94, IMAC E119, IMAC E20, IMAC E45, IMAC E70, IMAC E95, IMAC E120, IMAC E21, IMAC E46, IMAC E71, IMAC E96, IMAC E121, IMAC E22, IMAC E47, IMAC E72, IMAC E97, IMAC E122, IMAC E23, IMAC E48, IMAC E73, IMAC E98, and IMAC E123. In some embodiments, the condition is selected from IMAC E143, IMAC E168, IMAC E193, IMAC E218, IMAC E243, IMAC E144, IMAC E169, IMAC E194, IMAC E219, IMAC E244, IMAC E145, IMAC E170, IMAC E195, IMAC E220, IMAC E245, IMAC E146, IMAC E171, IMAC E196, IMAC E221, IMAC E246, IMAC E147, IMAC E172, IMAC E197, IMAC E222, and IMAC E247.

In some embodiments, the IMAC comprises any one of the following washing/elution condition combinations:
(1) IMAC washing condition of any one of IMAC W1 to IMAC W25, and IMAC elution condition of any one of IMAC E51 to IMAC E75;
(2) IMAC washing condition of any one of IMAC W1 to IMAC W25, and IMAC elution condition of any one of IMAC E126 to IMAC E150;
(3) IMAC washing condition of any one of IMAC W1 to IMAC W25, and IMAC elution condition of any one of IMAC E226 to IMAC E250;
(4) IMAC washing condition of any one of IMAC W76 to IMAC W100, and IMAC elution condition of any one of IMAC E51 to IMAC E75;
(5) IMAC washing condition of any one of IMAC W76 to IMAC W100, and IMAC elution condition of any one of IMAC E126 to IMAC E150;
(6) IMAC washing condition of any one of IMAC W76 to IMAC W100, and IMAC elution condition of any one of IMAC E226 to IMAC E250;
(7) IMAC washing condition of any one of IMAC W176 to IMAC W200, and IMAC elution condition of any one of IMAC E51 to IMAC E75;
(8) IMAC washing condition of any one of IMAC W176 to IMAC W200, and IMAC elution condition of any one of IMAC E126 to IMAC E150;
(9) IMAC washing condition of any one of IMAC W176 to IMAC W200, and IMAC elution condition of any one of IMAC E226 to IMAC E250.

The IMAC step as described herein can be either performed under refrigerated conditions (e.g., 8±3° C.), or under ambient temperature.

In some embodiments, a method as described herein comprises both a CEX chromatography and an IMAC, either in tandem or separately, regardless of the order of the CEX chromatography and the IMAC, wherein the method comprises any one of the following wash/elution conditions:
(1) CEX washing condition of any one of CEX W1 to CEX W25, and CEX elution condition of any one of CEX E1 to CEX E25; IMAC washing condition of any one of IMAC W1 to IMAC W25, and IMAC elution condition of any one of IMAC E51 to IMAC E75;
(2) CEX washing condition of any one of CEX W1 to CEX W25, and CEX elution condition of any one of CEX E1 to CEX E25; IMAC washing condition of any one of IMAC W1 to IMAC W25, and IMAC elution condition of any one of IMAC E126 to IMAC E150;
(3) CEX washing condition of any one of CEX W1 to CEX W25, and CEX elution condition of any one of CEX E1 to CEX E25; IMAC washing condition of any one of IMAC W1 to IMAC W25, and IMAC elution condition of any one of IMAC E226 to IMAC E250;
(4) CEX washing condition of any one of CEX W1 to CEX W25, and CEX elution condition of any one of CEX E1 to CEX E25; IMAC washing condition of any one of IMAC W76 to IMAC W100, and IMAC elution condition of any one of IMAC E51 to IMAC E75;
(5) CEX washing condition of any one of CEX W1 to CEX W25, and CEX elution condition of any one of CEX E1 to CEX E25; IMAC washing condition of any one of IMAC W76 to IMAC W100, and IMAC elution condition of any one of IMAC E126 to IMAC E150;
(6) CEX washing condition of any one of CEX W1 to CEX W25, and CEX elution condition of any one of CEX E1 to CEX E25; IMAC washing condition of any one of IMAC W76 to IMAC W100, and IMAC elution condition of any one of IMAC E226 to IMAC E250;
(7) CEX washing condition of any one of CEX W1 to CEX W25, and CEX elution condition of any one of CEX E1 to CEX E25; IMAC washing condition of any one of IMAC W176 to IMAC W200, and IMAC elution condition of any one of IMAC E51 to IMAC E75;
(8) CEX washing condition of any one of CEX W1 to CEX W25, and CEX elution condition of any one of CEX E1 to CEX E25; CEX washing condition of any one of CEX W1 to CEX W25, and CEX elution condition of any one of CEX E1 to CEX E25; IMAC washing condition of any one of IMAC W176 to IMAC W200, and IMAC elution condition of any one of IMAC E126 to IMAC E150;
(9) CEX washing condition of any one of CEX W1 to CEX W25, and CEX elution condition of any one of CEX E1 to CEX E25; IMAC washing condition of any one of IMAC W176 to IMAC W200, and IMAC elution condition of any one of IMAC E226 to IMAC E250.
(10) CEX washing condition of any one of CEX W1 to CEX W25, and CEX elution condition of any one of CEX E101 to CEX E125;
(11) IMAC washing condition of any one of IMAC W1 to IMAC W25, and IMAC elution condition of any one of IMAC E51 to IMAC E75;
(12) CEX washing condition of any one of CEX W1 to CEX W25, and CEX elution condition of any one of CEX E101 to CEX E125; IMAC washing condition of any one of IMAC W1 to IMAC W25, and IMAC elution condition of any one of IMAC E126 to IMAC E150;
(13) CEX washing condition of any one of CEX W1 to CEX W25, and CEX elution condition of any one of CEX E101 to CEX E125; IMAC washing condition of any one of IMAC W1 to IMAC W25, and IMAC elution condition of any one of IMAC E226 to IMAC E250;
(14) CEX washing condition of any one of CEX W1 to CEX W25, and CEX elution condition of any one of CEX E101 to CEX E125; IMAC washing condition of any one of IMAC W76 to IMAC W100, and IMAC elution condition of any one of IMAC E51 to IMAC E75;
(15) CEX washing condition of any one of CEX W1 to CEX W25, and CEX elution condition of any one of CEX E101 to CEX E125; IMAC washing condition of any one of IMAC W76 to IMAC W100, and IMAC elution condition of any one of IMAC E126 to IMAC E150;
(16) CEX washing condition of any one of CEX W1 to CEX W25, and CEX elution condition of any one of CEX E101 to CEX E125; IMAC washing condition of any one of IMAC W76 to IMAC W100, and IMAC elution condition of any one of IMAC E226 to IMAC E250;
(17) CEX washing condition of any one of CEX W1 to CEX W25, and CEX elution condition of any one of CEX E101 to CEX E125; IMAC washing condition of any one of IMAC W176 to IMAC W200, and IMAC elution condition of any one of IMAC E51 to IMAC E75;
(18) CEX washing condition of any one of CEX W1 to CEX W25, and CEX elution condition of any one of CEX E101 to CEX E125; IMAC washing condition of any one of IMAC W176 to IMAC W200, and IMAC elution condition of any one of IMAC E126 to IMAC E150;
(19) CEX washing condition of any one of CEX W1 to CEX W25, and CEX elution condition of any one of CEX E101 to CEX E125; IMAC washing condition of any one of IMAC W176 to IMAC W200, and IMAC elution condition of any one of IMAC E226 to IMAC E250.
(20) CEX washing condition of any one of CEX W1 to CEX W25, and CEX elution condition of any one of CEX E226 to CEX E250; IMAC washing condition of any one of IMAC W1 to IMAC W25, and IMAC elution condition of any one of IMAC E51 to IMAC E75;
(21) CEX washing condition of any one of CEX W1 to CEX W25, and CEX elution condition of any one of CEX E226 to CEX E250; IMAC washing condition of any one of IMAC W1 to IMAC W25, and IMAC elution condition of any one of IMAC E126 to IMAC E150;
(22) CEX washing condition of any one of CEX W1 to CEX W25, and CEX elution condition of any one of CEX E226 to CEX E250; IMAC washing condition of any one of IMAC W1 to IMAC W25, and IMAC elution condition of any one of IMAC E226 to IMAC E250;
(23) CEX washing condition of any one of CEX W1 to CEX W25, and CEX elution condition of any one of CEX E226 to CEX E250; IMAC washing condition of any one of IMAC W76 to IMAC W100, and IMAC elution condition of any one of IMAC E51 to IMAC E75;
(24) CEX washing condition of any one of CEX W1 to CEX W25, and CEX elution condition of any one of CEX E226 to CEX E250; IMAC washing condition of any one of IMAC W76 to IMAC W100, and IMAC elution condition of any one of IMAC E126 to IMAC E150;
(25) CEX washing condition of any one of CEX W1 to CEX W25, and CEX elution condition of any one of CEX E226 to CEX E250; IMAC washing condition of any one of IMAC W76 to IMAC W100, and IMAC elution condition of any one of IMAC E226 to IMAC E250;
(26) CEX washing condition of any one of CEX W1 to CEX W25, and CEX elution condition of any one of CEX E226 to CEX E250; IMAC washing condition of any one of IMAC W176 to IMAC W200, and IMAC elution condition of any one of IMAC E51 to IMAC E75;
(27) CEX washing condition of any one of CEX W1 to CEX W25, and CEX elution condition of any one of CEX E226 to CEX E250; IMAC washing condition of any one of IMAC W176 to IMAC W200, and IMAC elution condition of any one of IMAC E126 to IMAC E150;
(28) CEX washing condition of any one of CEX W1 to CEX W25, and CEX elution condition of any one of CEX E226 to CEX E250; IMAC washing condition of any one of IMAC W176 to IMAC W200, and IMAC elution condition of any one of IMAC E226 to IMAC E250.
(29) CEX washing condition of any one of CEX W101 to CEX W125, and CEX elution condition of any one of CEX E1 to CEX E25; IMAC washing condition of any one of IMAC W1 to IMAC W25, and IMAC elution condition of any one of IMAC E51 to IMAC E75;
(30) CEX washing condition of any one of CEX W101 to CEX W125, and CEX elution condition of any one of CEX E1 to CEX E25; IMAC washing condition of any one of IMAC W1 to IMAC W25, and IMAC elution condition of any one of IMAC E126 to IMAC E150;
(31) CEX washing condition of any one of CEX W101 to CEX W125, and CEX elution condition of any one of CEX E1 to CEX E25; IMAC washing condition of any one of IMAC W1 to IMAC W25, and IMAC elution condition of any one of IMAC E226 to IMAC E250;
(32) CEX washing condition of any one of CEX W101 to CEX W125, and CEX elution condition of any one of CEX E1 to CEX E25; IMAC washing condition of any one of IMAC W76 to IMAC W100, and IMAC elution condition of any one of IMAC E51 to IMAC E75;
(33) CEX washing condition of any one of CEX W101 to CEX W125, and CEX elution condition of any one of CEX E1 to CEX E25; IMAC washing condition of any one of IMAC W76 to IMAC W100, and IMAC elution condition of any one of IMAC E126 to IMAC E150;
(34) CEX washing condition of any one of CEX W101 to CEX W125, and CEX elution condition of any one of CEX E1 to CEX E25; IMAC washing condition of any one of IMAC W76 to IMAC W100, and IMAC elution condition of any one of IMAC E226 to IMAC E250;
(35) CEX washing condition of any one of CEX W101 to CEX W125, and CEX elution condition of any one of CEX E1 to CEX E25; IMAC washing condition of any one of IMAC W176 to IMAC W200, and IMAC elution condition of any one of IMAC E51 to IMAC E75;
(36) CEX washing condition of any one of CEX W101 to CEX W125, and CEX elution condition of any one of CEX E1 to CEX E25; IMAC washing condition of any one of IMAC W176 to IMAC W200, and IMAC elution condition of any one of IMAC E126 to IMAC E150;
(37) CEX washing condition of any one of CEX W101 to CEX W125, and CEX elution condition of any one of CEX E1 to CEX E25; IMAC washing condition of any one of IMAC W176 to IMAC W200, and IMAC elution condition of any one of IMAC E226 to IMAC E250.
(38) CEX washing condition of any one of CEX W101 to CEX W125, and CEX elution condition of any one of CEX E101 to CEX E125; IMAC washing condition of any one of IMAC W1 to IMAC W25, and IMAC elution condition of any one of IMAC E51 to IMAC E75;
(39) CEX washing condition of any one of CEX W101 to CEX W125, and CEX elution condition of any one of CEX E101 to CEX E125; IMAC washing condition of any one of IMAC W1 to IMAC W25, and IMAC elution condition of any one of IMAC E126 to IMAC E150;
(40) CEX washing condition of any one of CEX W101 to CEX W125, and CEX elution condition of any one of CEX E101 to CEX E125; IMAC washing condition of any one of IMAC W1 to IMAC W25, and IMAC elution condition of any one of IMAC E226 to IMAC E250;
(41) CEX washing condition of any one of CEX W101 to CEX W125, and CEX elution condition of any one of CEX E101 to CEX E125; IMAC washing condition of any one of IMAC W76 to IMAC W100, and IMAC elution condition of any one of IMAC E51 to IMAC E75;
(42) CEX washing condition of any one of CEX W101 to CEX W125, and CEX elution condition of any one of CEX E101 to CEX E125; IMAC washing condition of any one of IMAC W76 to IMAC W100, and IMAC elution condition of any one of IMAC E126 to IMAC E150;
(43) CEX washing condition of any one of CEX W101 to CEX W125, and CEX elution condition of any one of CEX E101 to CEX E125; IMAC washing condition of any one of IMAC W76 to IMAC W100, and IMAC elution condition of any one of IMAC E226 to IMAC E250;
(44) CEX washing condition of any one of CEX W101 to CEX W125, and CEX elution condition of any one of CEX E101 to CEX E125; IMAC washing condition of any one of IMAC W176 to IMAC W200, and IMAC elution condition of any one of IMAC E51 to IMAC E75;
(45) CEX washing condition of any one of CEX W101 to CEX W125, and CEX elution condition of any one of CEX E101 to CEX E125; IMAC washing condition of any one of IMAC W176 to IMAC W200, and IMAC elution condition of any one of IMAC E126 to IMAC E150;
(46) CEX washing condition of any one of CEX W101 to CEX W125, and CEX elution condition of any one of CEX E101 to CEX E125; IMAC washing condition of any one of IMAC W176 to IMAC W200, and IMAC elution condition of any one of IMAC E226 to IMAC E250.
(47) CEX washing condition of any one of CEX W101 to CEX W125, and CEX elution condition of any one of CEX E226 to CEX E250; IMAC washing condition of any one of IMAC W1 to IMAC W25, and IMAC elution condition of any one of IMAC E51 to IMAC E75;
(48) CEX washing condition of any one of CEX W101 to CEX W125, and CEX elution condition of any one of CEX E226 to CEX E250; IMAC washing condition of any one of IMAC W1 to IMAC W25, and IMAC elution condition of any one of IMAC E126 to IMAC E150;
(49) CEX washing condition of any one of CEX W101 to CEX W125, and CEX elution condition of any one of CEX E226 to CEX E250; IMAC washing condition of any one of IMAC W1 to IMAC W25, and IMAC elution condition of any one of IMAC E226 to IMAC E250;
(50) CEX washing condition of any one of CEX W101 to CEX W125, and CEX elution condition of any one of CEX E226 to CEX E250; IMAC washing condition of any one of IMAC W76 to IMAC W100, and IMAC elution condition of any one of IMAC E51 to IMAC E75;
(51) CEX washing condition of any one of CEX W101 to CEX W125, and CEX elution condition of any one of CEX E226 to CEX E250; IMAC washing condition of any one of IMAC W76 to IMAC W100, and IMAC elution condition of any one of IMAC E126 to IMAC E150;
(52) CEX washing condition of any one of CEX W101 to CEX W125, and CEX elution condition of any one of CEX E226 to CEX E250; IMAC washing condition of any one of IMAC W76 to IMAC W100, and IMAC elution condition of any one of IMAC E226 to IMAC E250;
(53) CEX washing condition of any one of CEX W101 to CEX W125, and CEX elution condition of any one of CEX E226 to CEX E250; IMAC washing condition of any one of IMAC W176 to IMAC W200, and IMAC elution condition of any one of IMAC E51 to IMAC E75;
(54) CEX washing condition of any one of CEX W101 to CEX W125, and CEX elution condition of any one of CEX E226 to CEX E250; IMAC washing condition of any one of IMAC W176 to IMAC W200, and IMAC elution condition of any one of IMAC E126 to IMAC E150;
(55) CEX washing condition of any one of CEX W101 to CEX W125, and CEX elution condition of any one of CEX E226 to CEX E250; IMAC washing condition of any one of IMAC W176 to IMAC W200, and IMAC elution condition of any one of IMAC E226 to IMAC E250.
(56) CEX washing condition of any one of CEX W226 to CEX W250, and CEX elution condition of any one of CEX E1 to CEX E25; IMAC washing condition of any one of IMAC W1 to IMAC W25, and IMAC elution condition of any one of IMAC E51 to IMAC E75;
(57) CEX washing condition of any one of CEX W226 to CEX W250, and CEX elution condition of any one of CEX E1 to CEX E25; IMAC washing condition of any one of IMAC W1 to IMAC W25, and IMAC elution condition of any one of IMAC E126 to IMAC E150;
(58) CEX washing condition of any one of CEX W226 to CEX W250, and CEX elution condition of any one of CEX E1 to CEX E25; IMAC washing condition of any one of IMAC W1 to IMAC W25, and IMAC elution condition of any one of IMAC E226 to IMAC E250;
(59) CEX washing condition of any one of CEX W226 to CEX W250, and CEX elution condition of any one of CEX E1 to CEX E25; IMAC washing condition of any one of IMAC W76 to IMAC W100, and IMAC elution condition of any one of IMAC E51 to IMAC E75;
(60) CEX washing condition of any one of CEX W226 to CEX W250, and CEX elution condition of any one of CEX E1 to CEX E25; IMAC washing condition of any one of IMAC W76 to IMAC W100, and IMAC elution condition of any one of IMAC E126 to IMAC E150;
(61) CEX washing condition of any one of CEX W226 to CEX W250, and CEX elution condition of any one of CEX E1 to CEX E25; IMAC washing condition of any one of IMAC W76 to IMAC W100, and IMAC elution condition of any one of IMAC E226 to IMAC E250;
(62) CEX washing condition of any one of CEX W226 to CEX W250, and CEX elution condition of any one of CEX E1 to CEX E25; IMAC washing condition of any one of IMAC W176 to IMAC W200, and IMAC elution condition of any one of IMAC E51 to IMAC E75;
(63) CEX washing condition of any one of CEX W226 to CEX W250, and CEX elution condition of any one of CEX E1 to CEX E25; IMAC washing condition of any one of IMAC W176 to IMAC W200, and IMAC elution condition of any one of IMAC E126 to IMAC E150;
(64) CEX washing condition of any one of CEX W226 to CEX W250, and CEX elution condition of any one of CEX E1 to CEX E25; IMAC washing condition of any one of IMAC W176 to IMAC W200, and IMAC elution condition of any one of IMAC E226 to IMAC E250.
(65) CEX washing condition of any one of CEX W226 to CEX W250, and CEX elution condition of any one of CEX E101 to CEX E125; IMAC washing condition of any one of IMAC W1 to IMAC W25, and IMAC elution condition of any one of IMAC E51 to IMAC E75;
(66) CEX washing condition of any one of CEX W226 to CEX W250, and CEX elution condition of any one of CEX E101 to CEX E125; IMAC washing condition of any one of IMAC W1 to IMAC W25, and IMAC elution condition of any one of IMAC E126 to IMAC E150;
(67) CEX washing condition of any one of CEX W226 to CEX W250, and CEX elution condition of any one of CEX E101 to CEX E125; IMAC washing condition of any one of IMAC W1 to IMAC W25, and IMAC elution condition of any one of IMAC E226 to IMAC E250;
(68) CEX washing condition of any one of CEX W226 to CEX W250, and CEX elution condition of any one of CEX E101 to CEX E125; IMAC washing condition of any one of IMAC W76 to IMAC W100, and IMAC elution condition of any one of IMAC E51 to IMAC E75;
(69) CEX washing condition of any one of CEX W226 to CEX W250, and CEX elution condition of any one of CEX E101 to CEX E125; IMAC washing condition of any one of IMAC W76 to IMAC W100, and IMAC elution condition of any one of IMAC E126 to IMAC E150;
(70) CEX washing condition of any one of CEX W226 to CEX W250, and CEX elution condition of any one of CEX E101 to CEX E125; IMAC washing condition of any one of IMAC W76 to IMAC W100, and IMAC elution condition of any one of IMAC E226 to IMAC E250;
(71) CEX washing condition of any one of CEX W226 to CEX W250, and CEX elution condition of any one of CEX E101 to CEX E125; IMAC washing condition of any one of IMAC W176 to IMAC W200, and IMAC elution condition of any one of IMAC E51 to IMAC E75;
(72) CEX washing condition of any one of CEX W226 to CEX W250, and CEX elution condition of any one of CEX E101 to CEX E125; IMAC washing condition of any one of IMAC W176 to IMAC W200, and IMAC elution condition of any one of IMAC E126 to IMAC E150;
(73) CEX washing condition of any one of CEX W226 to CEX W250, and CEX elution condition of any one of CEX E101 to CEX E125; IMAC washing condition of any one of IMAC W176 to IMAC W200, and IMAC elution condition of any one of IMAC E226 to IMAC E250;
(74) CEX washing condition of any one of CEX W226 to CEX W250, and CEX elution condition of any one of CEX E226 to CEX E250; IMAC washing condition of any one of IMAC W1 to IMAC W25, and IMAC elution condition of any one of IMAC E51 to IMAC E75;
(75) CEX washing condition of any one of CEX W226 to CEX W250, and CEX elution condition of any one of CEX E226 to CEX E250; IMAC washing condition of any one of IMAC W1 to IMAC W25, and IMAC elution condition of any one of IMAC E126 to IMAC E150;
(76) CEX washing condition of any one of CEX W226 to CEX W250, and CEX elution condition of any one of CEX E226 to CEX E250; IMAC washing condition of any one of IMAC W1 to IMAC W25, and IMAC elution condition of any one of IMAC E226 to IMAC E250;
(77) CEX washing condition of any one of CEX W226 to CEX W250, and CEX elution condition of any one of CEX E226 to CEX E250; IMAC washing condition of any one of IMAC W76 to IMAC W100, and IMAC elution condition of any one of IMAC E51 to IMAC E75;
(78) CEX washing condition of any one of CEX W226 to CEX W250, and CEX elution condition of any one of CEX E226 to CEX E250; IMAC washing condition of any one of IMAC W76 to IMAC W100, and IMAC elution condition of any one of IMAC E126 to IMAC E150;
(79) CEX washing condition of any one of CEX W226 to CEX W250, and CEX elution condition of any one of CEX E226 to CEX E250; IMAC washing condition of any one of IMAC W76 to IMAC W100, and IMAC elution condition of any one of IMAC E226 to IMAC E250;
(80) CEX washing condition of any one of CEX W226 to CEX W250, and CEX elution condition of any one of CEX E226 to CEX E250; IMAC washing condition of any one of IMAC W176 to IMAC W200, and IMAC elution condition of any one of IMAC E51 to IMAC E75;
(81) CEX washing condition of any one of CEX W226 to CEX W250, and CEX elution condition of any one of CEX E226 to CEX E250; IMAC washing condition of any one of IMAC W176 to IMAC W200, and IMAC elution condition of any one of IMAC E126 to IMAC E150;
(82) CEX washing condition of any one of CEX W226 to CEX W250, and CEX elution condition of any one of CEX E226 to CEX E250; IMAC washing condition of any one of IMAC W176 to IMAC W200, and IMAC elution condition of any one of IMAC E226 to IMAC E250.

### Recombinant Acid Sphingomyelinase Compositions

The present disclosure provides compositions comprising recombinant acid sphingomyelinase (rASM). In some embodiments, the rASM is recombinant human acid sphingomyelinase (rhASM). In some embodiments, the rhASM is olipudase alfa. In some embodiments, the rhASM comprises a polypeptide having SEQ ID NO: 1, 2, 3, 4, 5, or 6. In some embodiments, the rhASM comprises a polypeptide having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more identity to any one of SEQ ID NOs: 1-6, or a mixture thereof.

In some embodiments, the compositions as described herein are pharmaceutical compositions. In some embodiments, the pharmaceutical compositions are formulated according to the methods described herein. In some embodiments, the compositions are in liquid formulation. In some embodiments, the compositions are lyophilized formulations.

In some embodiments, the composition is a rASM preparation, such as a rhASM preparation. In some embodiments, the preparation is a final product ready for therapeutic use or commercial sale. In some embodiments, the preparation is an intermediate product for downstream manufacture.

In some embodiments, the present disclosure provides a container (e.g., a vial) containing the composition described herein.

In some embodiments, the compositions of the present disclosure contain rhASM and demonstrate superior rhASM isoform uniformity and purity, with a well-controlled specific activity.

In some embodiments, the rASM compositions of the present disclosure have a purity of at least 80%, at least 81%, at least 82%, at least 83%, at least 84%, at least 85%, at least 86%, at least 87%, at least 88%, at least 90%, at least 91%, at least 92%, at least 93%, at least 94%, at least 95.0%, at least 95.5%, at least 96.0%, at least 96.5%, at least 97.0%, at least 97.5%, at least 98.0%, at least 98.5%, at least 99%, at least 99.5% or more. Purity of a composition of the present disclosure can be determined by suitable methods known in the art. In some embodiments, the purity is determined by HPLC, such as RP-HPLC.

In some embodiments, the rASM compositions of the present disclosure have an unmodified rASM isoform that is at least 50%, at least 55%, at least 60%, at least 65%, at least 70%, at least 75%, at least 80%, at least 85%, at least 90%, at least 95% or more of the total rASM population in the rASM preparation. In some embodiments, the unmodified rASM isoform in the rASM composition is at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more of the total rASM population in the rASM preparation. In some embodiments, all modified rASM isoforms in total are no more than 50%, no more than 40%, no more than 35%, no more than 30%, no more than 25%, no more than 20%, no more than 15%, no more than 14%, no more than 13%, no more than 12%, no more than 11%, no more than 10%, no more than 9%, no more than 8%, no more than 7%, no more than 6%, no more than 5% or less of the total rASM population in the rASM preparation.

In some embodiments, the rASM isoform having C-terminus cysteinylation is no more than 50%, no more than 40%, no more than 35%, no more than 30%, no more than 25%, no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5% or less of the total rASM population in the rASM preparation.

In some embodiments, the rASM isoform having C-terminus S-glutathionylation is no more than 50%, no more than 40%, no more than 35%, no more than 30%, no more than 25%, no more than 20%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, or no more than 1% of the total rASM population in the rASM preparation. In some embodiments, the rASM isoform having C-terminus S-glutathionylation is no more than 5%, no more than 4%, no more than 3%, no more than 2%, no more than 1% or less of the total rASM population in the rASM preparation.

In some embodiments, the rASM isoform having C-terminus dimerization is no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3%, no more than 2%, no more than 1%, no more than 0.9%, no more than 0.8%, no more than 0.7%, no more than 0.6%, no more than 0.5%, no more than 0.4%, no more than 0.3%, or no more than 0.2% of the total rASM population in the rASM preparation. In some embodiments, the rASM isoform having C-terminus dimerization is no more than 0.1% of the total rASM population in the rASM preparation. Isoforms having C-terminus dimerization include, but are not limited to, those described in Table 1.

In some embodiments, the rASM isoforms having C-terminus truncation are no more than 50%, no more than 40%, no more than 35%, no more than 30%, no more than 25%, no more than 20%, no more than 15%, no more than 14%, no more than 13%, no more than 12%, no more than 11%, no more than 10%, no more than 9%, no more than 8% of the total rASM population in the rASM preparation. In some embodiments, the rASM isoforms having C-terminus truncation are no more than 7%, no more than 6%, no more than 5%, no more than 4%, no more than 3%, no more than 2%, no more than 1%, or less of the total rASM population in the rASM preparation. Isoforms having C-terminus truncation include, but are not limited to, those described in Table 1.

Specific activity of the rASM compositions is about 5 U/mg, about 6 U/mg, about 7 U/mg, about 8 U/mg, about 9 U/mg, about 10 U/mg, about 11 U/mg, about 12 U/mg, about 13 U/mg, about 14 U/mg, about 15 U/mg, about 16 U/mg, about 17 U/mg, about 18 U/mg, about 19 U/mg, about 20 U/mg, about 21 U/mg, about 22 U/mg, about 23 U/mg, about 24 U/mg, about 25 U/mg, about 26 U/mg, about 27 U/mg, about 28 U/mg, about 29 U/mg, about 30 U/mg, about 31 U/mg, about 32 U/mg, about 33 U/mg, about 34 U/mg, about 35 U/mg, about 36 U/mg, about 37 U/mg, about 38 U/mg, about 39 U/mg, about 40 U/mg, about 41 U/mg, about 42 U/mg, about 43 U/mg, about 44 U/mg, about 45 U/mg, about 46 U/mg, about 47 U/mg, about 48 U/mg, about 49 U/mg, or about 50 U/mg. In some embodiments, the specific activity is about 5 to 50 U/mg. In some embodiments, the specific activity is about 10 to 40 U/mg. In some embodiments, the specific activity is about 10 to 30 U/mg. In some embodiments, the specific activity is about 10 to 20 U/mg. In some embodiments, specific activity in the obtained rASM preparation is about 5-50 U/mg, such as about 10-40 U/mg, about 15-45 U/mg, about 10-30 U/mg, about 15-35 U/mg, or about 10-20 U/mg. In some embodiments, specific activity in the obtained rASM preparation is about 5 U/mg, about 10 U/mg, about 15 U/mg, about 20 U/mg, about 25 U/mg, about 30 U/mg, about 35 U/mg, about 40 U/mg, about 45 U/mg, or about 50 U/mg. Specific activity of the rASM compositions can be determined by a suitable method known in the art. In some embodiments, specific activity of the rASM composition is determined by the assay as described in Example 4.

Recombinant ASM compositions as described herein have a host cell protein (HCP) level no more than 5.0 μg/mg. In some embodiments, the rASM compositions have a HCP level no more than 5.0 μg/mg, no more than 4.5 μg/mg, no more than 4.0 μg/mg, no more than 3.5 μg/mg, no more than 3.0 μg/mg, no more than 2.5 μg/mg, no more than 2.0 g/mg, no more than 1.5 μg/mg, no more than 1.0 μg/mg, no more than 0.9 μg/mg, no more than 0.8 μg/mg, no more than 0.7 μg/mg, no more than 0.6 μg/mg, no more than 0.5 μg/mg, or less.

In some embodiments, rASM compositions of the present disclosure may have at least one, at least two, at least three, at least four, at least five, at least six, at least seven, or at least eight characteristics selected from the group consisting of:
(1) having a purity of at least 80%, at least 81%, at least 82%, at least 83%, at least 84%, at least 85%, at least 86%, at least 87%, at least 88%, at least 90%, at least 91%, at least 92%, at least 93%, at least 94%, at least 95.0%, at least 95.5%, at least 96.0%, at least 96.5%, at least 97.0%, at least 97.5%, at least 98.0%, at least 98.5%, at least 99%, at least 99.5% or more;
(2) having an unmodified rASM isoform in the rASM composition that is at least 50%, at least 55%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more of the total rASM population;
(3) having an rASM isoform comprising C-terminus cysteinylation that is no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5% or less of the total rASM population;
(4) having an rASM isoform comprising C-terminus S-glutathionylation that is no more than 10%, no more than 9%, no more than 8%, no more than 7%, no more than 6%, no more than 5%, no more than 4%, no more than 3%, no more than 2%, no more than 1% or less of the total rASM population;
(5) having an rASM isoform comprising C-terminus dimerization that is no more than 2%, no more than 1.9%, no more than 1.8%, no more than 1.7%, no more than 1.6%, no more than 1.5%, no more than 1.4%, no more than 1.3%, no more than 1.2%, no more than 1.1%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1% of the total rASM population;
(6) having an rASM isoform comprising a C-terminus truncation that is no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, no more than 5%, no more than 4%, no more than 3% or less than the total rASM population;
(7) having a specific activity that is about 10 to 50 U/mg, about 15 to 35 U/mg, about 10 to 30 U/mg, or about 10 to 20 U/mg; and
(8) having a host cell protein (HCP) level no more than 5.0 μg/mg, no more than 4.5 g/mg, no more than 4.0 μg/mg, no more than 3.5 μg/mg, no more than 2.0 μg/mg, no more than 1.5 μg/mg, no more than 1.0 μg/mg, no more than 0.5 μg/mg, or less;
In some embodiments, the compositions comprising recombinant acid sphingomyelinase (rASM) as described herein are produced by purifying rASM expressed in host cells, using the methods as described herein. Particularly, compositions comprising rASM as described herein are produced through a process comprising a purification method as described herein. In further embodiments, compositions comprising rASM as described herein are purified using a CEX chromatography as descried herein. In further embodiments, compositions comprising rASM as described herein are purified using a CEX chromatography and/or an IMAC as descried herein.
   Formulated Pharmaceutical Composition Comprising Purified rhASM

The present disclosure also provides pharmaceutical compositions comprising purified rASM (e.g., rhASM) as described herein. In some embodiments, the pharmaceutical compositions are made by formulating an rASM preparation as described herein. In some embodiments, the formulation process does not change, or does not significantly change the specific activity of rASM in the rASM preparation and/or the relevant ratio of rASM isoforms in the preparation. For example, after the formulation process, the specific activity of rASM in the composition is at least 80%, at least 85%, at least 90%, at least 95%, at least 96%, at least 97%, at least 98%, at least 99%, at least 1×, at least 2×, at least 3×, at least 4×, at least 5×, at least 6×, at least 7×, at least 8×, at least 9×, at least 10×, at least 15×, at least 20×, at least 30×, at least 40×, at least 50× or more compared to the specific activity before the formulation process.

In some embodiments, the compositions of the present invention contain one or more pharmaceutically acceptable excipients. “Excipient” refers to an inert substance that is used as a diluent, vehicle, carrier, preservative, binder, or stabilizing agent for the active ingredient(s) of a drug. For example, the compositions may contain a buffering agent, an isotonic agent, and/or a stabilizing agent such as an anti-oxidant. In some cases, one agent may serve more than one of these purposes. In some embodiments, a composition of the invention contains a recombinant human ASM such as olipudase alfa, a buffering agent such as sodium phosphate or sodium citrate, a stabilizer such as L-methionine, and a nonreducing sugar such as sucrose or trehalose. The human ASM has improved stability due to the particular makeup in the composition. The compositions of the invention may be aqueous liquid solutions or lyophilized preparations.

In some embodiments, the composition is an aqueous liquid composition comprising 1-10 mg/mL (e.g., 3-5 mg/mL) rhASM (e.g., olipudase alfa); 10-50 mM (e.g., 10-30 mM) sodium phosphate; 70-150 mM (e.g., 80-120 mM) methionine (e.g., L-methionine); and 1-10% (e.g., 4-6%) w/v sucrose or trehalose. The pH of the aqueous liquid composition may be 5-8 (e.g., 6-7).

In some embodiments, the aqueous liquid composition comprises no detectable amount of mannitol, the most readily used crystalline excipient, because it may significantly increase aggregation of the human ASM during or after the lyophilization of an aqueous liquid composition described herein.

In some embodiments, the aqueous liquid composition comprises 0.004-0.008%, 0.005-0.007%, or 0.005% w/v surfactant(s). Exemplary surfactants include nonionic detergents, such as polysorbates (e.g., polysorbates 20 and 80) and poloxamers (e.g., poloxamer 188). In a particular embodiment, the aqueous liquid composition comprises 0.005% polysorbate 80. In some cases, the presence of surfactant(s) may help to reduce turbidity in the liquid composition.

In some embodiments, the aqueous liquid composition comprises no more than 0.05, 0.01, or 0.005 mM chelating agent(s), such as EDTA and EGTA; in an exemplary embodiment, the aqueous liquid composition comprises no detectable amount of chelating agent(s). In some cases, the presence of chelating agents at a concentration above, e.g., 0.05 mM or 0.1 mM, may increase aggregation of the human ASM and decrease its stability, particularly after a prolonged storage period, e.g., for 12-16 weeks, or under non-refrigerated conditions, e.g., at 25° C. In some embodiments, the aqueous liquid composition may contain 0-50 ppm (e.g., 15-30 ppm) of zinc, which may be, e.g., carried over from the manufacturing process or added externally.

In a particular embodiment, the aqueous liquid composition comprises or consists essentially of 4 mg/mL olipudase alfa, 20 mM sodium phosphate, 100 mM methionine, and 5% (w/v) sucrose and has a pH of 6.5. The term “consists essentially of” means that the composition does not contain other ingredients at detectable amounts or may contain only trace amounts of certain materials that are derived from the protein manufacturing process where such materials do not affect the biological activity of the enzyme or cause harm in human patients.

In some embodiments, the composition is an aqueous liquid composition comprising 1-20 mg/mL (e.g., 10 mg/mL) rhASM (e.g., olipudase alfa) and 10-50 mM (e.g., 20 mM) sodium phosphate. In certain embodiments, the aqueous liquid composition further comprises methionine (e.g., L-methionine) and sucrose or trehalose. In certain embodiments, the aqueous liquid composition further comprises 80-120 mM (e.g., 100 mM) methionine and 4-6% (e.g., 5%) (w/v) sucrose. In particular embodiments, the aqueous liquid composition has a pH of 6.5.

In some embodiments, the composition is an aqueous liquid composition comprising 1-50 mg/mL (e.g., 3.8, 18, or 49 mg/mL) rhASM (e.g., olipudase alfa) and 10-50 mM (e.g., 20 mM) sodium phosphate. In certain embodiments, the aqueous liquid composition further comprises 1-15% (e.g., 5%, 6%, 7%, or 8%) sucrose or trehalose. In certain embodiments, the aqueous liquid composition further comprises 80-120 mM (e.g., 100 mM) methionine. In particular embodiments, the aqueous liquid composition has a pH of 6.5. The composition may comprise, for example, 3.8 mg/mL rhASM, 20 mM sodium phosphate, and 5% sucrose; 18 mg/mL rhASM, 20 mM sodium phosphate, and 5% sucrose; or 49 mg/mL rhASM, 20 mM phosphate, and 8% sucrose.

The aqueous liquid compositions may be prepared by mixing a human ASM produced by recombinant technology and subsequently purified from host cells with excipients described herein in water, and adjusting the resulting mixture to the desired pH. For example, the human ASM and desired excipients may be added to, or buffer-exchanged into, a sodium phosphate buffer with the desired sodium phosphate concentration and pH.

In some embodiments, the aqueous liquid composition may be prepared by reconstituting a lyophilized composition of the invention further described in detail below. The reconstitution may be done with a pharmaceutically acceptable liquid such as sterile water, saline (e.g., 0.9% sodium chloride), or phosphate-buffered saline.

The present invention also provides lyophilized compositions. Such compositions can be prepared by lyophilizing the aqueous liquid compositions described herein. Lyophilized compositions are suitable for long term storage. Lyophilization may be performed according to methods known in the art. For example, a liquid composition may be cooled to a subzero (Celsius) temperature (e.g., −5° C. to −80° C.) that allows freezing, and then placed in a low pressure (partial vacuum) chamber to allow sublimation to occur (primary drying); where desired, the temperature of the composition may be raised in a second stage of drying (secondary drying) to further remove unwanted water molecules. In some embodiments, after completion of the lyophilization process, an inert gas such as nitrogen may be introduced into the container of the composition (e.g., a glass vial) before the container is sealed.

In some embodiments, the present invention provides powdered compositions, which may be prepared, e.g., by spray-drying the aqueous liquid compositions described herein. Spray-dried compositions are suitable for long term storage. Spray-drying may be performed according to methods known in the art. For example, a liquid composition may be forced through an atomizer or spray nozzle to disperse it as controlled-size tiny droplets into a hot gas stream in a chamber, resulting in rapid drying of the liquid composition to powder. The dried powder may then be collected at the bottom of the drying chamber. Other drying methods for preparing powdered compositions are also contemplated.

Sucrose (or trehalose) and methionine present at amounts described herein provide superior results during lyophilization; the lyophilized products form elegant cakes while preserving the stability of the human ASM during storage. The human ASM in the lyophilized compositions of the present invention may remain free of aggregation and biologically active for at least 4 months (e.g., at least 6 months or at least 12 months) under refrigerated conditions (e.g., at 0-10° C., 2-8° C., or 4° C.).

In some embodiments, the composition of the invention is a lyophilized pharmaceutical composition comprising 4-50% olipudase alfa, 3-7% sodium phosphate, and 45-90% sucrose (all w/w percentages). In certain embodiments, the lyophilized composition comprises 5.5% olipudase alfa, 20.6% L-methionine, 2.3% sodium phosphate dibasic heptahydrate, 2.6% sodium phosphate monobasic monohydrate, and 69.0% sucrose (all w/w percentages). In certain embodiments, the lyophilized composition comprises 6.6% olipudase alfa, 3.0% sodium phosphate dibasic heptahydrate, 3.3% sodium phosphate monobasic monohydrate, and 87.1% sucrose (all w/w percentages). In certain embodiments, the lyophilized composition comprises 25.2% olipudase alfa, 2.4% sodium phosphate dibasic heptahydrate, 2.6% sodium phosphate monobasic monohydrate, and 69.9% sucrose (all w/w percentages). In certain embodiments, the lyophilized composition comprises 47.8% olipudase alfa, 1.7% sodium phosphate dibasic heptahydrate, 1.8% sodium phosphate monobasic monohydrate, and 48.8% sucrose (all w/w percentages).

In some embodiments, the composition of the invention is a lyophilized pharmaceutical composition comprising 4-7% olipudase alfa, 15-25% L-methionine, 3-7% sodium phosphate, and 65-75% sucrose (all w/w percentages). In a particular embodiment, the lyophilized composition comprises 5.5% olipudase alfa, 20.5% L-methionine, 2.3% sodium phosphate dibasic heptahydrate, 2.6% sodium phosphate monobasic monohydrate, and 68.6% sucrose (all w/w percentages). In certain embodiments, the lyophilized composition may also comprise, e.g., 0.1%, 0.2%, 0.3%, 0.4%, 0.5%, 0.6%, 0.7%, 0.8%, 0.9%, or 1.0% moisture.

In some embodiments, the invention provides a vial containing a lyophilized pharmaceutical composition comprising 15-25 mg olipudase alfa, 75-85 mg L-methionine, 15-25 mg sodium phosphate, and 250-300 mg sucrose. Prior to use, the composition may be reconstituted in 4-6 mL of sterile water.

In some embodiments, the vial contains a lyophilized pharmaceutical composition comprising or consisting of 21.2 mg, 20.1 mg, 95.4 mg, or 259.7 mg of olipudase alfa; 9.0 mg sodium phosphate dibasic heptahydrate; 10.0 mg sodium phosphate monobasic monohydrate; and 265 mg sucrose. The lyophilized composition may optionally comprise 79.1 mg L-methionine. The lyophilized pharmaceutical composition may optionally comprise 0-0.3 mg (e.g., 0.08-0.16 mg) zinc, which may be, e.g., carried over from the manufacturing process or added externally. In certain embodiments, the vial may have an internally sterile nitrogen filled atmosphere. In a particular embodiment, the lyophilized composition may be reconstituted in 5.1 mL of sterile water to yield an olipudase alfa concentration of about 4.0 mg/mL, 3.8 mg/mL, 18 mg/mL, or 49 mg/mL, respectively. The reconstituted composition may be further diluted in 0.9% sodium chloride solution to a specific volume based on the dose to be administered.

In a particular embodiment, the vial contains a lyophilized pharmaceutical composition comprising or consisting of 21.2 mg olipudase alfa, 79 mg L-methionine, 9.0 mg sodium phosphate dibasic heptahydrate, 10.0 mg sodium phosphate monobasic monohydrate, and 265 mg sucrose. The lyophilized pharmaceutical composition may optionally comprise 0-0.3 mg (e.g., 0.08-0.16 mg) zinc, which may be, e.g., carried over from the manufacturing process or added externally. In certain embodiments, the lyophilized pharmaceutical composition is in the form of a cake or a lyophilized powder. In certain embodiments, the vial may have an internally sterile nitrogen filled atmosphere. In a particular embodiment, the lyophilized composition may be reconstituted in 5.1 mL of sterile water to yield an olipudase alfa concentration of about 4.0 mg/mL. The reconstituted composition may be further diluted in 0.9% sodium chloride solution to a specific volume based on the dose to be administered.

In some embodiments, the invention provides a vial containing a lyophilized pharmaceutical composition comprising 3-5 mg olipudase alfa, 15-17 mg L-methionine, 3-5 mg sodium phosphate, and 50-60 mg sucrose. Prior to use, the composition may be reconstituted in 0.8-1.2 mL of sterile water.

In a particular embodiment, the vial contains a lyophilized pharmaceutical composition comprising or consisting of 4.8 mg olipudase alfa, 17.9 mg L-methionine, 2.0 mg sodium phosphate dibasic heptahydrate, 2.3 mg sodium phosphate monobasic monohydrate, and 60 mg sucrose. In certain embodiments, the lyophilized pharmaceutical composition is in the form of a cake or a lyophilized powder. The lyophilized composition may optionally comprise 0-0.06 mg zinc, which may be, e.g., carried over from the manufacturing process or added externally. In certain embodiments, the vial may have an internally sterile nitrogen filled atmosphere. In a particular embodiment, the lyophilized composition may be reconstituted in 1.1 mL of sterile water to yield an olipudase alfa concentration of about 4.0 mg/mL. The reconstituted composition may be further diluted in 0.9% sodium chloride solution to a specific volume based on the dose to be administered.

### Articles of Manufacture

rASM purified by the methods described herein and/or formulations comprising the rASM purified by the methods described herein may be contained within an article of manufacture. The article of manufacture may comprise a container containing the rASM and/or the rASM formulation. In certain embodiments, the article of manufacture comprises: (a) a container comprising a composition comprising the rASM and/or the rASM formulation described herein within the container; and (b) a package insert with instructions for administering the formulation to a subject.

The article of manufacture comprises a container and a label or package insert on or associated with the container. Suitable containers include, for example, bottles, vials, syringes, etc. The containers may be formed from a variety of materials such as glass or plastic. The container holds or contains a formulation and may have a sterile access port (for example the container may be an intravenous solution bag or a vial having a stopper pierceable by a hypodermic injection needle). At least one active agent in the composition is the polypeptide. The label or package insert indicates that the composition's use in a subject with specific guidance regarding dosing amounts and intervals of polypeptide and any other drug being provided. The article of manufacture may further include other materials desirable from a commercial and user standpoint, including other buffers, diluents, filters, needles, and syringes. In some embodiments, the container is a syringe. In some embodiments, the syringe is further contained within an injection device. In some embodiments, the injection device is an autoinjector.

A “package insert” is used to refer to instructions customarily included in commercial packages of therapeutic products, that contain information about the indications, usage, dosage, administration, contraindications, other therapeutic products to be combined with the packaged product, and/or warnings concerning the use of such therapeutic products.

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for.

### Example 1: Impact of Purification on rhASM Specific Activity

During process characterization studies, efforts were made in order to better understand the factors which impact rhASM specific activity. Objectives of such process characterization studies were to understand how manufacturing process impacts rhASM product specific activity, and to find critical steps that are sufficient to control specific activity of the product.

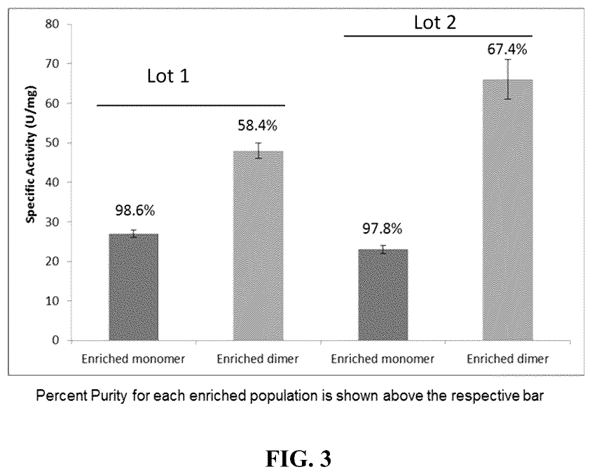

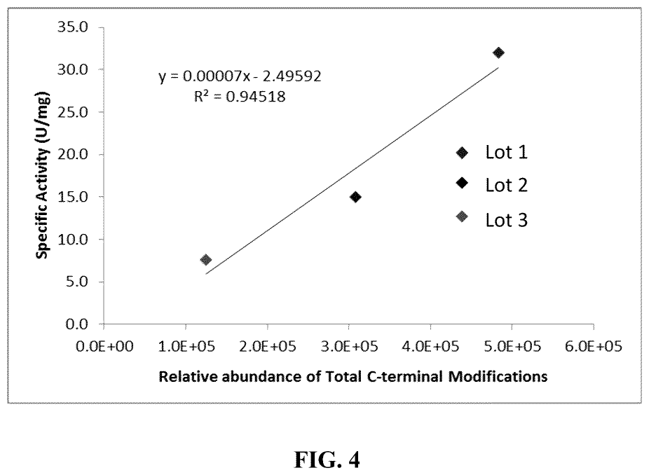

Several different isoforms of rhASM exist in the ASM population produced in a bioreactor. Details of the rhASM isoforms are described in Table 1. Studies have identified that dimerized forms of rhASM possess higher specific activity as compared to the monomeric form of rhASM (FIG. 3). Also, isoforms of olipudase alfa that have been chemically modified at a C-terminal Cysteine (C570) possess higher specific activity than the unmodified form, possibly due to associations between C570 and the active site. The studies further demonstrate a correlation between abundance of C-terminal modified forms and increased specific activity (FIG. 4). Essentially, if there is a higher abundance of modified rhASM in a composition, the composition would have higher specific activity.

A sample comprising rhASM was subjected to a cation exchange (CEX) chromatography. rhASM binds to the resin while impurities flow through. Further impurity reduction was achieved with a post-load wash, and olipudase alfa was then eluted from the column by increasing the salt concentration compared to that of the wash buffer.

The cation exchange (CEX) chromatography eluates were further purified by immobilized metal affinity chromatography (IMAC). The resin was charged with metal ions. The cation exchange (CEX) chromatography eluate was adjusted to a target pH and loaded onto the equilibrated IMAC column. rhASM was then eluted with a high salt elution buffer.

The immobilized metal affinity chromatography (IMAC) eluate was collected, mixed, and concentrated. The obtained bulk sample was then formulated as described in Example 2.

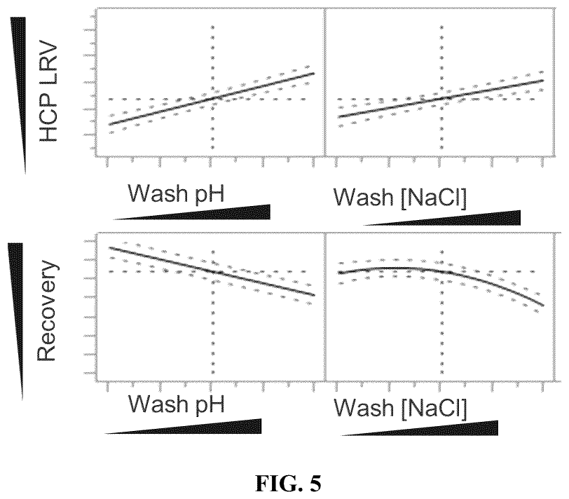

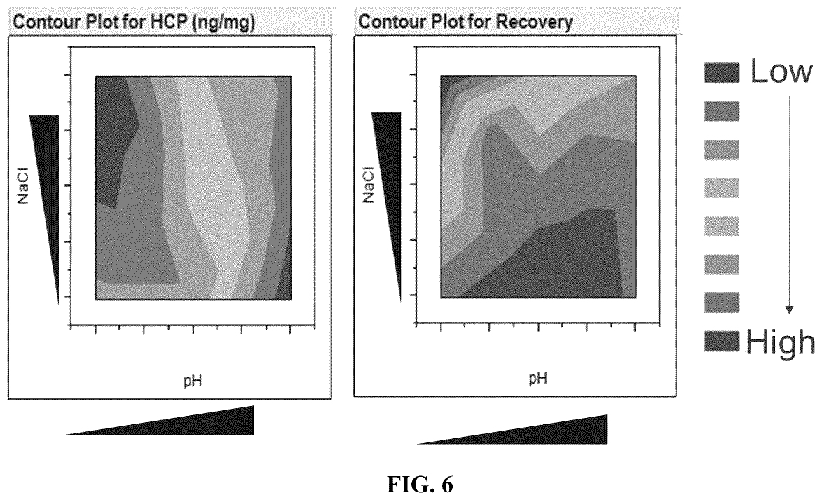

It was discovered that purity (as measured by RP-HPLC) of the rhASM preparation was well controlled in the CEX chromatography and/or the IMAC step. As demonstrated in FIG. 5, in the CEX chromatography step, greater HCP clearance was achieved at higher pH, higher salt concentrations, but greater HCP clearance led to a lower recovery yield. Similarly, in the IMAC step, as demonstrated in FIG. 6, greater HCP clearance was achieved at lower pH, higher salt concentrations, but greater HCP clearance also led to a lower yield. Recovery rate was measured by both A280 absorbance and activity level. HCP level was measured both by Octet® assay and ELISA.

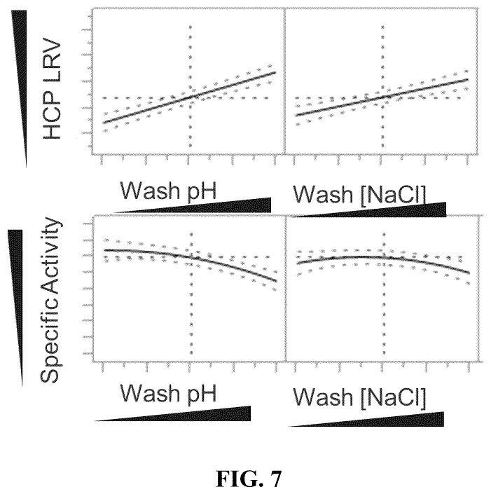

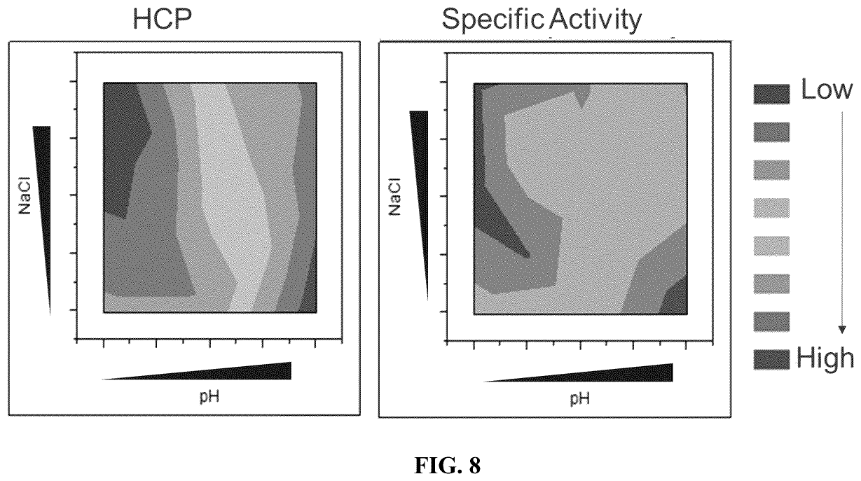

Surprisingly, conditions which resulted in superior HCP clearance had adverse impact on specific activity. As demonstrated in FIG. 7, in the CEX chromatography step, although greater HCP clearance was achieved at higher pH, higher salt conditions, the obtained rhASM preparation had lower specific activity. Similarly, in the IMAC step, as demonstrated in FIG. 8, although greater HCP clearance was achieved at lower pH, higher salt conditions, the obtained rhASM preparation had lower specific activity.

The inventors hypothesized that highly active rhASM isoforms were weakly bound to the chromatography columns and were lost in the wash steps of the chromatography steps.

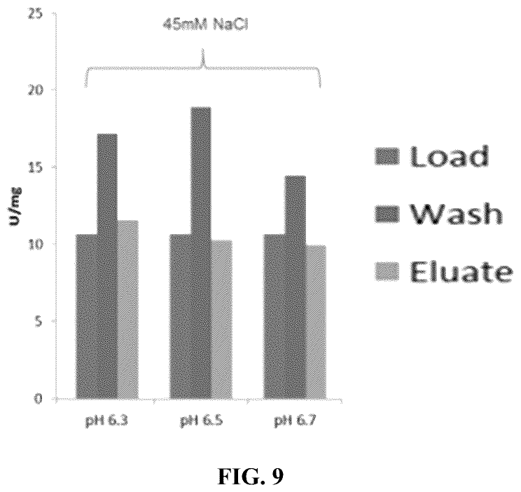

To test this hypothesis, in the CEX chromatography step, load material (e.g., product pool—rhASM samples before CEX chromatography step), wash fractions obtained in the CEX chromatography step, and eluate fractions obtained in the CEX chromatography step were analyzed to determine their specific activity in vitro as described in Example 4. Indeed, wash fractions collected from the CEX operation exhibited greater specific activity than the initial load material and the eluate fractions (FIG. 9, at the specified wash conditions). Several other salt concentration/pH washing conditions were also tested which led to similar results.

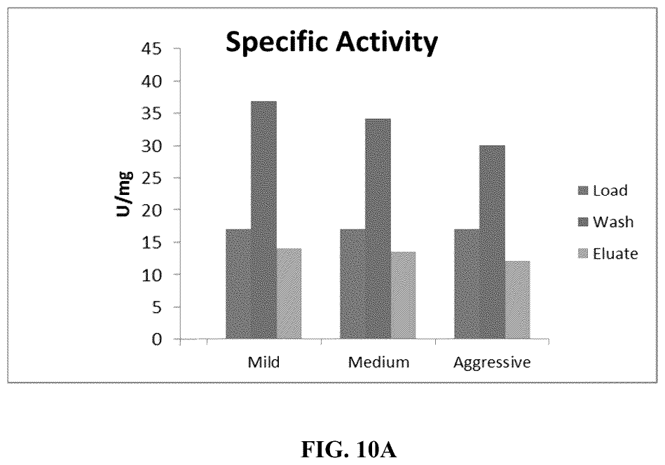

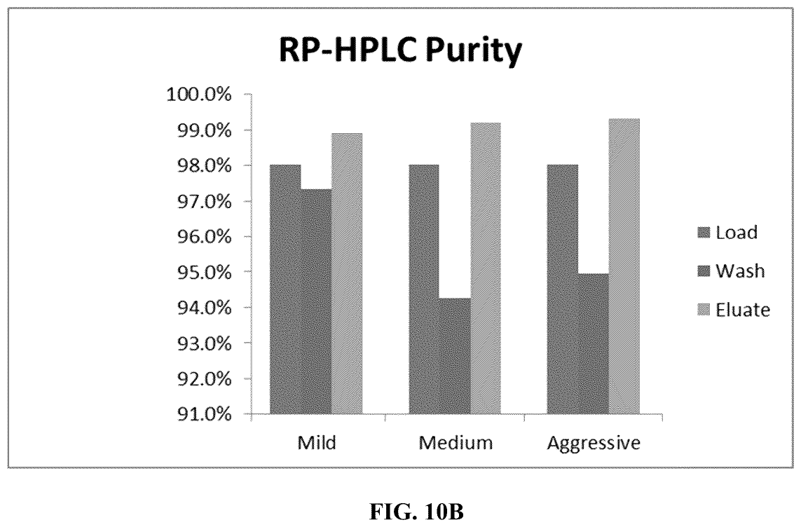

In the IMAC step, load material (e.g., product pool—rhASM samples before IMAC chromatography step), wash fractions obtained in the IMAC step, and eluate fractions obtained in the IMAC step were also analyzed to determine their specific activity in vitro. Similarly, wash fractions collected from the IMAC operation exhibited greater specific activity than the initial load material and the eluate fractions (FIG. 10A, at the specified wash conditions). FIG. 10B shows that the IMAC operation further improved purity of rhASM in the eluate fractions. A purity of over 98% or 99% was achieved. Several other salt concentrations/pH conditions were also tested which led to similar results.

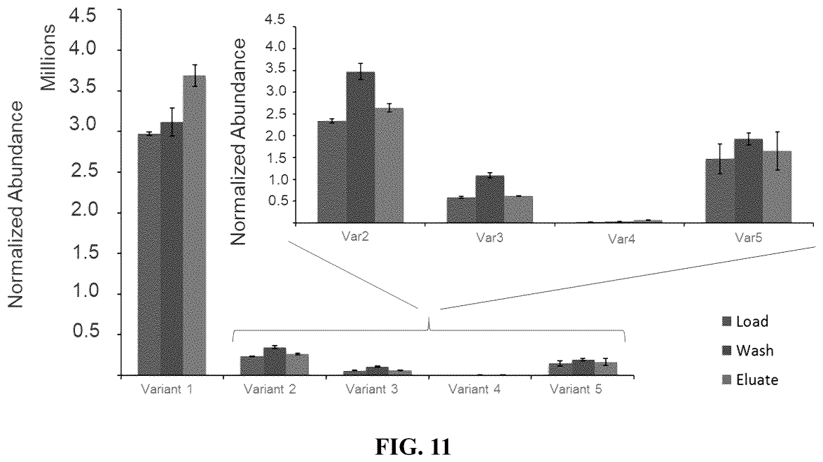

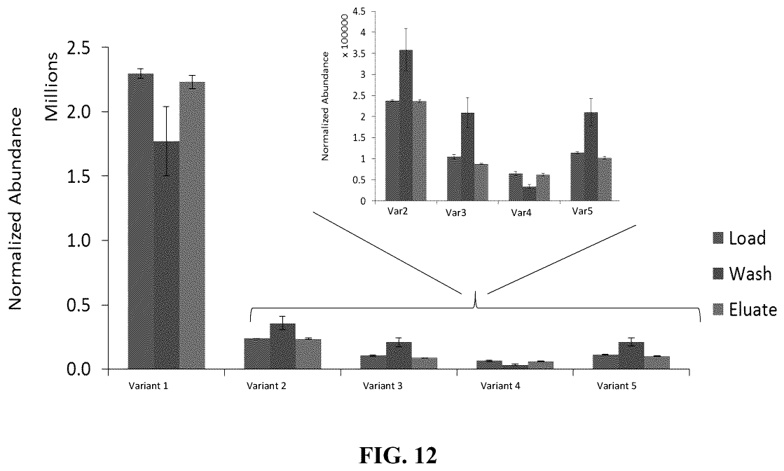

To further verify, load material, wash fractions, and eluate fractions of the CEX operation and the IMAC operation were submitted for mass spectrometry analysis to quantify the unmodified isoform and the modified isoforms. Free cysteine residue of rhASM was labeled before the ASM isoform progeny was analyzed. Relative abundances of the unmodified isoform and the modified isoforms are depicted in FIG. 11 (CEX operation) and FIG. 12 (IMAC operation). Indeed, in both operations, rhASM isoforms with C-terminal modifications were enriched in the wash fraction.

It was also observed that through the CEX operation, the relative abundances of the unmodified rhASM isoform and the modified rhASM isoforms were changed in both the wash fractions and the eluate fractions. LC-MS analysis showed that the relative abundances of the unmodified rhASM isoform in the eluate fraction was enriched compared to the load material. The modified rhASM isoforms together in the eluate population were lower compared to the load material. Thus, the CEX operation is capable of modulating the ratio between unmodified rhASM isoforms and modified rhASM isoforms in the rhASM product.

In summary, optimization of two bind-and-elute chromatography steps in the manufacturing process of olipudase alfa resulted in an unexpected impact on specific activity and rhASM isoform population. Specific activity was observed to decrease across both operations, and high specific activity was observed in the wash fractions for both operations. Mass spectrometry identified weakly bound “activated” rhASM isoforms which were enriched in the wash fraction and reduced in the eluate fractions. The analytical results for both studies suggest that specific activity in the eluates of either the CEX or the IMAC operations can be lowered by selective removal of highly active C-terminal modified species during the wash steps. In addition, the operations can modulate the ratio between unmodified rhASM isoforms and modified rhASM isoforms in the rhASM preparation, thereby improving the uniformity of rhASM population in the final product. The process as described herein allows for modulation of the specific activity and ratio of unmodified rhASM isoforms and modified rhASM isoforms during the manufacturing process, thereby to obtain an rhASM preparation having desired specific activity.

### Example 2: Recombinant Human Acid Sphingomyelinase Formulation

The formulation step is performed to achieve the final olipudase alfa and excipient concentrations in drug substance.

The formulation is performed according to the method described in WO 2019/227029 A1, which is herein incorporated by reference in its entirety. rhASM preparation obtained from the purification process in Example 1 was filtered to remove viral contamination, and concentrated to increase protein concentration prior to formulation. The formulated bulk was mixed and the rhASM concentration was determined by measuring absorbance at 280 nm. The product pool was then further diluted into a target volume to obtain the final drug substance olipudase alfa concentration.

Optionally, the liquid composition obtained herein can be spray-dried. Spray-dried compositions are suitable for long term storage. Spray-drying may be performed according to methods known in the art. For example, a liquid composition may be forced through an atomizer or spray nozzle to disperse it as controlled-size tiny droplets into a hot gas stream in a chamber, resulting in rapid drying of the liquid composition to powder. The dried powder may then be collected at the bottom of the drying chamber. Other drying methods for preparing powdered compositions are also contemplated.

### Example 3: Efficacy, Safety, Pharmacodynamic, and Pharmacokinetics Study of Olipudase Alfa in Patients With Acid Sphingomyelinase Deficiency

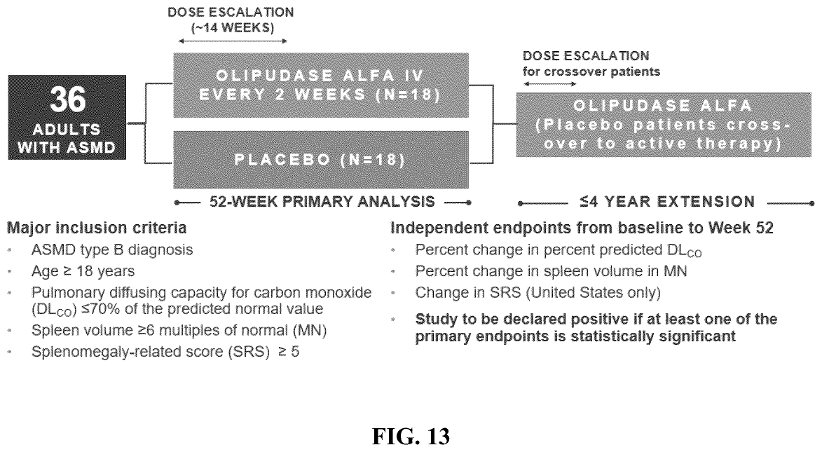

The recombinant human olipudase alfa formulation prepared in Example 2 was used in a clinical study to evaluate its efficacy. The study design is demonstrated in FIG. 13. Baseline patient and disease characteristics are provided in Table 6.

TABLE 6
Baseline Patient and Disease Characteristics

| | Placebo | Olipudase alfa |
|---|---|---|
| Parameter | (N = 18) | (N = 18) |
| Mean age at baseline in years (range) | 33.5 (18.6-65.9) | 36.2 (18.8-59.9) |
| Mean age at diagnosis in years ± SD | 14.6 ± 16.1 | 21.4 ± 20.3 |
| Sex, male/female, n (%) | 5 (28%)/13 (72%) | 9 (50%)/9 (50%) |
| Mean % predicted DL_{CO} adjusted for | 48.5 ± 10.8 | 49.4 ± 11.0 |
| hemoglobin ± SD | | |
| Mean spleen volume in MN ± SD | 11.21 ± 3.84 | 11.70 ± 4.92 |
| Mean splenomegaly-related score | 28.05 ± 10.6 | 24.55 ± 11.1 |
| (SRS) ± SD | | |
| Mean liver volume in MN ± SD | 1.62 ± 0.50 | 1.44 ± 0.32 |
| Mean platelet count × 10⁹/L ± SD | 115.6 ± 36.3 | 107.2 ± 26.9 |

DL_{CO} = diffusing capacity for carbon monoxide;
MN = multiples of normal;
SD = standard deviation;
SRS = Splenomegaly-Related Score

The primary objective of this study was to evaluate the efficacy of the formulation administered intravenously once every 2 weeks for 52 weeks in adult patients with acid sphingomyelinase deficiency (ASMD) by assessing changes in: 1) spleen volume as measured by abdominal magnetic resonance imaging (MRI) (and, for the United States [US] only, in association with patient perception related to spleen volume as measured by splenomegaly related score [SRS]); and 2) infiltrative lung disease as measured by the pulmonary function test, diffusing capacity of the lung for carbon monoxide (DLCO).

### Primary Endpoint—Percent Change in % Predicted DL_{CO}

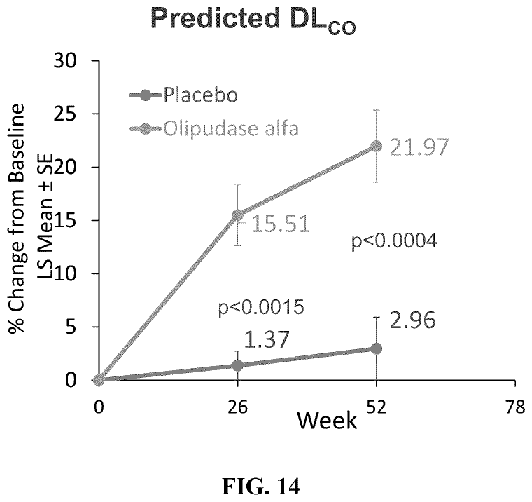

The results as shown in FIG. 14 indicate that at baseline, mean % predicted DL_{CO} was similar and reflected moderate disease in both groups; mean improvement from baseline to week 52 was 22% for the olipudase alfa group vs 3% for the placebo group; difference between groups was 19% at Week 52 (p<0.0004), and improvement was seen as early as Week 26; the study is declared positive; and the mean % predicted FVC also improved in the olipudase alfa group but not the placebo group at Week 52.

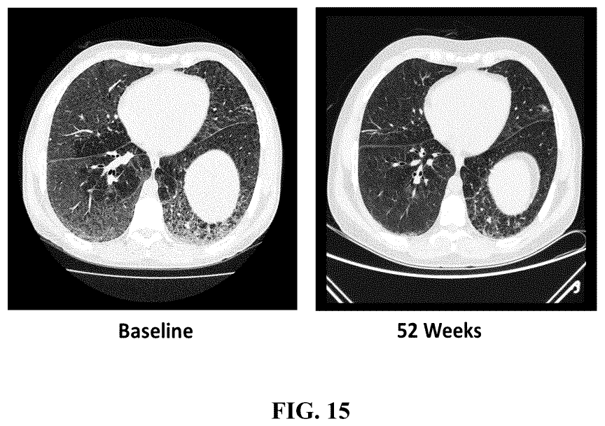

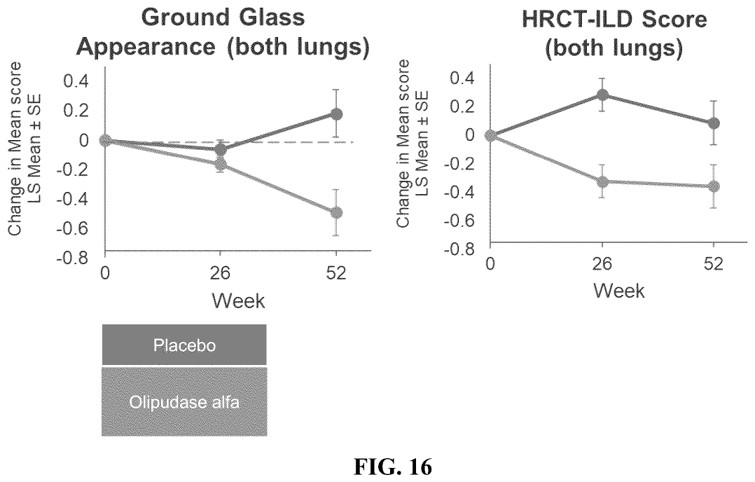

Pulmonary imaging studies also showed improvement in ASMD-mediated interstitial lung disease. An illustrative high-resolution computerized tomography image from an olipudase alfa-treated patient (FIG. 15) shows clearance of “ground glass” opacities caused by sphingomyelin-filled macrophages. HRCT ground glass appearance scores and interstitial lung disease scores in both lungs showed mean improvements in olipudase alfa treated but not placebo treated patients (FIG. 16).

### Primary Endpoint—Spleen Response

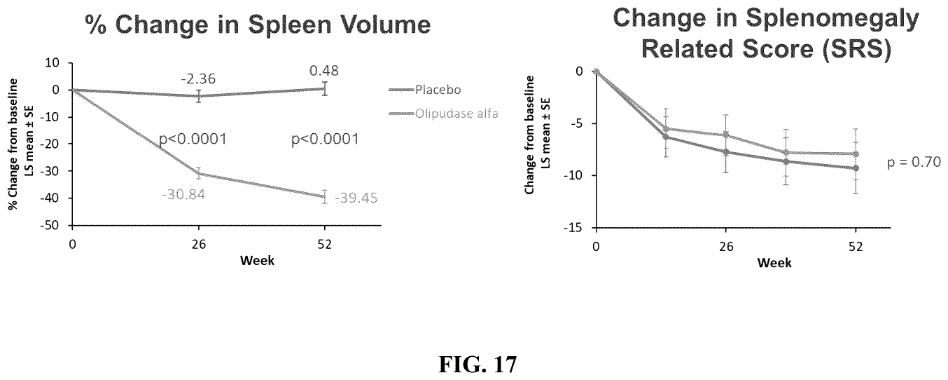

Spleen volume decreased in all olipudase alfa treated patients but not in placebo treated patients (FIG. 17, left panel). Mean baseline spleen volume was 11.2 MN (multiples of normal) in the placebo group and 11.7 MN in the olipudase group, signifying moderate splenomegaly. In olipudase alfa treated patients, substantial reduction in spleen volume was seen by 6 months of treatment and the largest reductions were seen in the patients who had the worst baseline splenomegaly. A statistically significant 39% reduction in spleen size in olipudase alfa treated patients vs 0.5% increase in placebo patients was demonstrated at Week 52 (p<0.0001). Furthermore, 17 out of 18 olipudase-alfa-treated patients (94%) had a decrease in spleen volume ≥30% vs 0/18 placebo patients. Largest reductions were seen in olipudase alfa treated patients with largest spleens at baseline.

Splenomegaly-related score was also calculated in olipudase alfa treated patients and in placebo treated patients, which decreased in parallel in both groups (FIG. 17, right panel). There was no correlation between SRS and baseline or final spleen volume; thus, symptoms measured by SRS did not reflect physiological disease burden. Mean baseline SRS scores were 28.1 for placebo and 24.6 for olipudase alfa. Both arms showed a reduction in SRS score; however, the LS mean change in SRS score from baseline to Week 52 was not statistically different in the olipudase alfa group (−7.66) compared to the placebo group (−9.28) after the multiplicity adjustment; p=0.6364. Of note, the SRS was adapted from myelofibrosis trials and was not previously validated in ASMD patients.

### Secondary Endpoint—Hepatic Response

Patients had moderate baseline hepatomegaly, with mean liver volumes of 1.6 MN in the placebo group and 1.4 MN in the olipudase alfa group, in addition to atherogenic lipid profiles and abnormal liver function tests.

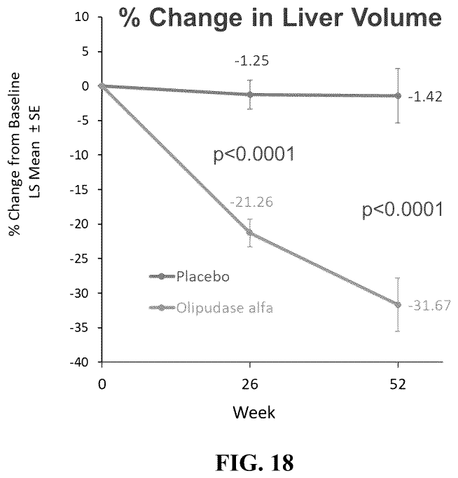

The LS mean percentage change in liver volume from baseline to Week 52 demonstrated greater reduction in the olipudase alfa group (31.67%) compared to the placebo group (1.42%, nominal p<0.0001). (FIG. 18). Similarly, the baseline atherogenic lipid profile improved in olipudase-alfa-treated patients but not placebo patients, with mean reductions in LDL cholesterol and triglycerides and increases in HDL cholesterol. Mean ALT and AST and other liver function tests also improved in olipudase-alfa but not placebo-treated patients.

### Secondary Endpoint—Platelet Count

Mean platelet counts improved in olipudase alfa- but not placebo-treated patients (+16.8% vs. +2.5%, respectively, p=0.019) with clinical differences seen by Week 26.

### Exploratory Endpoint—Liver Sphingomyelin

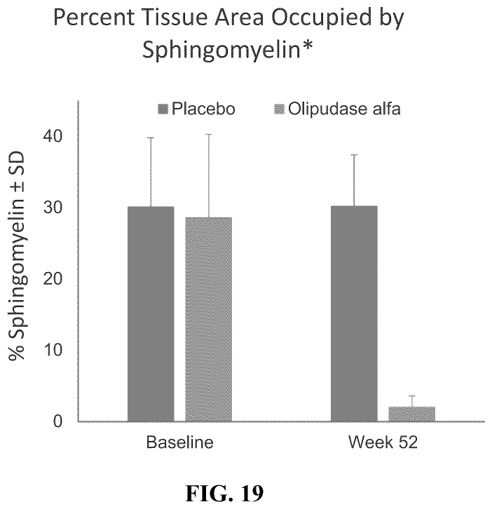

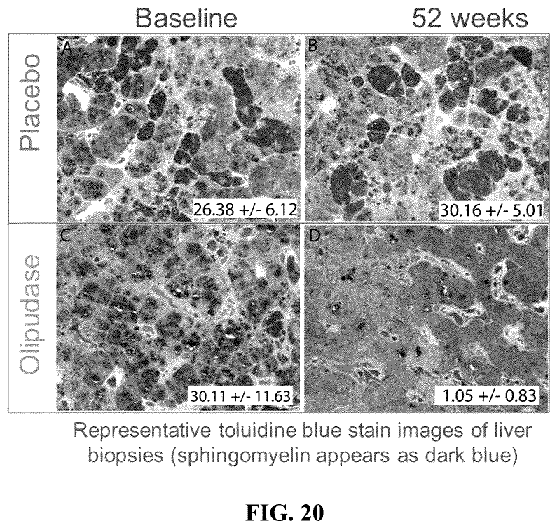

Liver sphingomyelin level was monitored in olipudase alfa treated patients and placebo treated patients. In olipudase alfa treated patients, the mean percent tissue area occupied by sphingomyelin decreased from 29% to 2% after 52 weeks but was unchanged in placebo patients (FIG. 19). Histological analysis of liver biopsy data showed substantial clearance of sphingomyelin in Kupffer cells and hepatocytes in olipudase alfa treated but not placebo treated patients, as demonstrated by the representative liver biopsy images (FIG. 20).

### Exploratory Endpoint—Biomarker Response

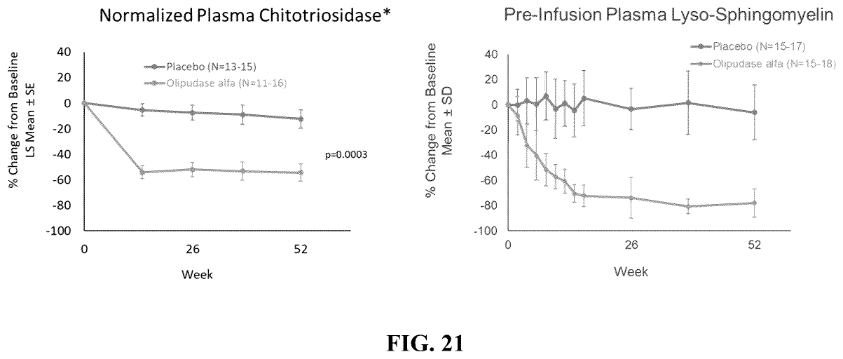

Mean levels of the plasma biomarkers chitotriosidase and lyso-sphingomyelin were both markedly elevated at baseline. Baseline values in both groups were: >14×ULN for chitotriosidase and >38×ULN for lyso-sphingomyelin. Mean chitotriosidase levels decreased by 54% in olipudase alfa group vs 12% for placebo at Week 52. Mean lyso-sphingomyelin decreased by 78% in olipudase alfa group vs 6% for placebo at Week 52. Both showed substantial reductions beginning with the first weeks of treatment in olipudase-alfa-treated but not placebo-treated patients (FIG. 21).

### Summary

The overall safety and tolerability profiles were favorable with no new safety risks identified in this trial. No patient died and there were no permanent discontinuations of olipudase alfa due to adverse events. All patients continued in the trial extension except one patient who discontinued during the primary analysis period due to poor compliance. 3 olipudase alfa treated patients had 5 serious adverse events and 4 placebo patients had 11 serious adverse events, but no serious adverse event was considered related to treatment. Infusion-associated reactions, which are expected in patients beginning enzyme replacement therapy, were mild or moderate and were easily managed. 4 out of the 18 olipudase alfa treated patients developed treatment-induced anti-drug antibodies; 2 of these patients had transient antibodies and the remaining 2 patients had persistent but low antibody titers. No patient developed neutralizing antibodies that interfered with cell uptake of enzymes.

The study is declared positive as it met the DL_{CO} endpoint and the totality of data demonstrate clinical benefit. ASMD-associated interstitial lung disease was improved in patients treated with the rhASM produced by the methods as described herein. Spleen volume was decreased, accompanied by increased platelet count, reflecting correction of hypersplenism. Liver volume was decreased due to clearance of sphingomyelin, supported by histological evidence from serial liver biopsies. Metabolic function was improved, as evidenced by improvement in liver function tests and lipid profile. Symptoms measured by SRS did not reflect disease burden as measured by physiological measures (spleen volume). The benefit-risk profile for olipudase alfa in adults with ASMD is favorable based on this study.

The clinical study suggests that rhASM compositions prepared by the method as described in the present disclosure showed excellent safety and efficacy in treating ASMD patients.

### Example 4: rhASM Specific Activity In Vitro Activity Assay

This example describes a method to determine the activity in U/mL and specific activity in U/mg of recombinant human acid sphingomyelinase (rhASM) in a sample based on the hydrolysis of 2-(N-hezxadecanoylamino)-4-nitrophenylphosphorylcholine (HDA-PC), a synthetic substrate. The rate of hydrolysis of the synthetic substrate catalyzed by rhASM was measured as follows.

Approximately 1 μg/mL rhASM was incubated with 1 mM 2-(N-hexadecanoylamino)-4-nitrophenylphosphorylcholine in 50 mM sodium acetate, 0.1 mM zinc acetate, and 0.25 mg/mL bovine serum albumin (BSA) at pH 5.3 in a 37.0° C. circulating water bath for 15 minutes. Essentially, 80 μL of the substrate was added to 20 μL of the enzyme to start the reaction. The reaction was stopped with the addition of 300 μL of a 0.1 M glycine, 0.1 M NaOH, 50% ethanol solution and the absorbance of the released 2-(N-hexadecanoylamino)-4-nitrophenol (HDA-NP) product was measured at 415 nm. One unit of activity was defined as the amount of enzyme required to hydrolyze one μmol of 2-(N-hexadecanoylamino-4-nitrophenyl) phosphorylcholine (HDA-PC) to 2-(N-hexadecanoylamino-4-nitrophenol (HDA-NP) per minute under the defined assay conditions. The specific activity (in U/mg) was calculated by dividing the enzyme activity results (in U/mL) by the corresponding rhASM protein concentration (in mg/mL).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean at least one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one member of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which at least one limitation, element, clause, or descriptive term, from at least one of the claims or from at least one relevant portion of the description, is introduced into another claim. For example, a claim that is dependent on another claim can be modified to include at least one of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

In the claims, as well as in the specification above, all transitional phrases such as “comprising,” “including,” “carrying,” “having,” “containing,” “involving,” “holding,” and “composed of,” are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111. 03.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any at least one of the claims. Where ranges are given, any value within the range may explicitly be excluded from any at least one of the claims. Any embodiment, element, feature, application, or aspect of the compositions and/or methods of the disclosure, can be excluded from any at least one claims. For purposes of brevity, all of the embodiments in which at least one elements, features, purposes, or aspects is excluded are not set forth explicitly herein.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control. AMENDMENTS TO THE CLAIMS

1. A method of purifying recombinant acid sphingomyelinase (rASM) comprising the steps of:
(i) subjecting a protein mixture comprising rASM and host cell proteins (HCPs) to a cation exchange (CEX) chromatography, or
subjecting a protein mixture comprising rASM and HCPs to an immobilized metal affinity chromatography (IMAC), or subjecting a protein mixture comprising rASM and HCPs to both a CEX chromatography and an IMAC; and
(ii) collecting eluate from the CEX chromatography or the IMAC, thereby obtaining a purified rASM preparation.

2 The method of claim 1, wherein
the protein mixture is subjected to a CEX chromatography and an IMAC in tandem, and eluate obtained from the CEX chromatography is subjected to the IMAC;
the protein mixture is subjected to an IMAC and a CEX chromatography in tandem, and eluate obtained from the IMAC is subjected to the CEX chromatography: or
the protein mixture is subjected to both the CEX chromatography and the IMAC separately, with one or more additional steps in between

3-4. (canceled)

5. The method of claim 1, where the protein mixture comprising rASM and HCPs is subjected to one or more additional purification columns before or after the mixture is subjected to the CEX chromatography or the IMAC.

6. The method of claim 1, further comprising
a step of inactivating and/or removing potential viral contaminants, and/or
a step to concentrate the purified rASM.

7 (canceled)

8 The method of claim 1, wherein the rASM is a recombinant human acid sphingomyelinase (rhASM), and optionally wherein the rASM comprises the amino acid sequence of SEQ ID NO: 1 or SEO ID NO: 2

9. The method of claim 1, wherein the protein mixture is obtained from Chinese Hamster Ovary (CHO) cells expressing the rASM.

10. (canceled)

11. The method of claim 1, wherein the cation exchange (CEX) chromatography comprises a resin selected from the group consisting of carboxymethyl (CM), sulfoethyl (SE), sulfopropyl (SP), phosphate (P) and sulfonate (S).

12. The method of claim 1, wherein the IMAC is a chelating resin, and optionally wherein the IMAC is performed with zinc, copper, or nickel

13. (canceled)

14. The method of claim 1, wherein the CEX chromatography comprises washing the CEX chromatography column with a CEX wash buffer having a first optimal pH and a first optimal salt concentration, wherein the first optimal pH and the first optimal salt concentration are predetermined depending on the resin and starting specific activity of the protein mixture, and optionally wherein the CEX chromatography further comprises eluting the CEX chromatography column with a CEX elution buffer having a second optimal pH and a second optimal salt concentration, wherein under the second optimal pH and the second optimal salt concentration rA SM bound to the CEX chromatography column after the washing step is removed from the column

15. (canceled)

16. The method of claim 1, wherein the IMAC comprises washing the IMAC column with at least one IMAC wash buffer having a third optimal pH and a third optimal salt concentration, wherein the third optimal pH and the third optimal salt concentration are predetermined depending on the resin and starting specific activity of the protein mixture, and optionally wherein the IMAC further comprises eluting the IMAC column with an IMAC elution buffer having a fourth optimal pH and a fourth optimal salt concentration, wherein under the fourth optimal pH and the fourth optimal salt concentration ASM bound to the IMAC column after the washing step is removed from the column.

17. (canceled)

18. The method of claim 1, wherein the purified rASM preparation has a specific activity of about 5 to 50 U/mg, optionally about 10 to 45 U/mg, or optionally about 10 to 20 U/mg.

19-20. (canceled)

21. The method of claim 1, wherein the purified rASM preparation has an HCP level not more than 1.0 μg/mg or not more than 5.0 μg/mg.

22. The method of claim 1, wherein the purified rASM preparation comprises rASM isoforms with one or more modifications selected from the group consisting of C-terminus cysteinylation, S-glutathionylation, dimerization, and truncation, optionally wherein the rASM isoforms with the modifications are in total no more than 5%, 10%, 15%, 20%, 25%, 30%, 35%, or 40% of the whole rASM population.

23. (canceled)

24. The method of claim 1, wherein the protein mixture is produced in a bioreactor having a production scale of at least 100L or at least 500L

25. (canceled)

26. A method of modulating the relative amounts of isoforms of recombinant acid sphingomyelinase (rASM) in an initial rASM composition, wherein the initial rASM composition comprises an unmodified rASM isoform, and at least one rASM isoform having one or more modifications selected from the group consisting of C-terminus cysteinylation, S-glutathionylation, dimerization, and truncation, wherein the method comprises
(i) subjecting the initial rASM composition to a cation exchange (CEX) chromatography, or
subjecting the initial rASM composition to an immobilized metal affinity chromatography (IMAC), or subjecting the initial rASM composition to both a CEX chromatography and an IMAC; and
(ii) collecting eluate from the CEX chromatography or the IMAC, thereby obtaining a purified rASM preparation.

27-45. (canceled)

46. A method of modulating recombinant acid sphingomyelinase (rASM) specific activity in a liquid composition comprising an unmodified rASM isoform, and at least one rASM isoform having one or more modifications selected from the group consisting of C-terminus cysteinylation, S-glutathionylation, dimerization, and truncation, wherein the method comprises:
(i) subjecting the liquid composition to a cation exchange (CEX) chromatography, or subjecting the liquid composition to an immobilized metal affinity chromatography (IMAC), or
subjecting the liquid composition to both a CEX chromatography and an IMAC; and
(ii) collecting the eluate from the CEX chromatography or the IMAC, thereby obtaining a purified rASM preparation.

47-64. (canceled)

65. A recombinant acid sphingomyelinase (rASM) preparation comprising an unmodified rASM isoform and at least one rASM isoform species having one or more modifications selected from the group consisting of C-terminus cysteinylation, S-glutathionylation, dimerization, and truncation, wherein the unmodified rASM isoform is at least 50%, 55%, 60%, 65%, 70%, 75%, 80%, 85%, 86%, 87%, 88%, 89%, 90%, 91%, 92% 93%, 94%, 95% or more of the total rASM population in the rASM preparation.

66. (canceled)

67. The rASM preparation of claim 65 er-elaim-66, wherein all modified rASM isoforms in total are no more than 40%, no more than 30%, no more than 25% no more than 20%, no more than 15%, no more than 14%, no more than 13%, no more than 12%, no more than 11%, no more than 10%, no more than 9%, no more than 8%, no more than 7%, no more than 6%, no more than 5% or less of the total rASM population in the rASM preparation.

68. (canceled)

69. The rASM preparation of claim 65, wherein the rASM isoform having C-terminus cysteinylation is no more than 10%, no more than 9%, no more than 8%, no more than 7%, no more than 6%, no more than 5% or less of the total rASM population in the rASM preparation.

70. (canceled)

71. The rASM preparation of claim 65, wherein the rASM isoform having C-terminus S-glutathionylation is no more than 5%, no more than 4%, no more than 3%, no more than 2%, no more than 1% or less of the total rASM population in the rASM preparation.

72. (canceled)

73. The rASM preparation of claim 65, wherein the rASM isoform having C-terminus dimerization is no more than 0.2% or no more than 0.1% of the total rASM population in the rASM preparation.

74. (canceled)

75. The rASM preparation of claim 65, wherein the rASM isoform having C-terminus truncation is no more than 8%, no more than 7% no more than 6%, no more than 5%, no more than 4%, no more than 3% or less of the total rASM population in the rASM preparation.

76. (canceled)

77. The rASM preparation of claim 65,
wherein the rASM preparation has a purity of at least 95%, at least 96%, at least 97%, at least 98%, at least 99%, or more.

78-81. (canceled)

82. An rASM preparation manufactured using the method of claim 1.

83. A pharmaceutical composition prepared by using the recombinant acid sphingomyelinase (rASM) preparation of claim 65.

84. A method of treating acid sphingomyelinase deficiency in a subject in need thereof, comprising administering the pharmaceutical composition of claim 83 to the subject.

85. The method of claim 1, further comprising a step to buffer exchange the purified rASM.

86. The method of claim 1, wherein the method is conducted partially or fully under (i) refrigerated condition at 8±° C. and/or (ii) ambient temperature.

87. (canceled)
